SEQUENCE LISTING (3 sequences)

SEQ ID NO: 1 - RNA, 1848 nt, unidentified
  source 1..1848: mol_type = other RNA; note = Sphaerica sp.
ccatgcatgtctaagtataagctgattatactgtgaaactgcgaatggctcattaaatcagttagagtttatttgatggtaccttactactcggataaccgtagtaaagctacagctaatacgtgcgcaaatcccgactcacgaagggacgtatttattagattcaagaccgaccgtgctcccgcacgtccttggtgaatcatggtaacttcacgaatcgcatggcctcgtgccggcgatgtttcattcaactttctgccctatcaactttcgacggtagtatagaggactaccgtggtagtaacgggtgacggaggattagggttcgattccggagagggagcctgagaaacggctaccacatccaaggaaggcagcaggcgcgcaaattacccaatcctgacacagggaggtagtgacaataaatatcatgcgatggccaatcggtcgtcgactggaatgagtacagtttaaatcccttaacgaggatccattggagggcaagtctggtgccagcagccgcggtaattccagctccaatagcgtatatttaagttgttgcagttaaaaagctcgtagttggatttcgggtgggcgccgccggtctcccattgggtatgtactggcgtggcccgccttgctgtcggggacgctctcccgggcttcactgctcgggaagcggagtcgacgacgttactttgagtaaattagagtgttcaaagcaagccatcgctctgaatatgatagcatgggataacacgacaggactctggcctatcgtgttggtctgtaggaccggagtaatgattaagagggacagtcgggggcattcgtattccgttgtcagaggtgaaattcttggatttacggaagacgaacatctgcgaaagcatttgccaaggatgttttcattgatcaagaacgaaagttgggggctcaaagacgattagataccgtcgtagtctcaaccataaacgatgccgactagggattggcggacgttctttcgatgactccgccagcacctcatgagaaatcaaagtttttgggttccggggggagtatggtcgcaaggctgaaacttaaaggaattgacggaagggcaccaccaggcgtggagcctgcggcttaatttgactcaacacgggaaaacttaccaggtccagacataggaaggattgacagattgagagctctttcttgattctatgggtggtggtgcatggccgttcttagttggtgggttgccttgtcaggttgattccggtaacgaacgagacctcagcctgctaaatagcgacgtctgcctgtgcagtcggcccgcttcttagagggactgttggcgtctagccaatggaagtttgaggcaataacaggtctgtgatgcccttagatgttctgggccgcacgcgcgctacactgatacgttcagcgagcccctgctccgaaaggtgtgggtaatcttgcaaaccgtatcgtgatggggatagaacattgcaattattgttcttcaacgaggaatgcctagtaagcgcgagtcatcaactcgcgttgattacgtccctgccctttgtacacaccgcccgtcgctcctaccgattgaacgtgctggtgaagcgttaggactggagcccggctctaggttctcctggtccgggacccgggaatttcgttgaaccctcccgtttagaggaaggagaagtcgtaacaaggtttccgtaggtgaacctgcggaaggatcattgaatccatcccctgccccccagcgaaccgtcccagtacccctgtcgggcgcccttggcggggcgcccgccagcggccgggccgtcgcccagtacgggcggcgcc

SEQ ID NO: 2 - RNA, 1793 nt, unidentified
  source 1..1793: mol_type = other RNA; note = Sphaerica sp.
ccatgcatgtctaagtataagctgattatactgtgaaactgcgaatggctcattaaatcagttagagtttatttgatggtaccttactactcggataaccgtagtaaagctacagctaatacgtgcgcaaatcccgactcacgaagggacgtatttattagattcaagaccgaccgtgctcccgcacgtccttggtgaatcatggtaacttcacgaatcgcatggcctcgtgccggcgatgtttcattcaactttctgccctatcaactttcgacggtagtatagaggactaccgtggtagtaacgggtgacggaggattagggttcgattccggagagggagcctgagaaacggctaccacatccaaggaaggcagcaggcgcgcaaattacccaatcctgacacagggaggtagtgacaataaatatcatgcgatggccaatcggtcgtcgactggaatgagtacagtttaaatcccttaacgaggatccattggagggcaagtctggtgccagcagccgcggtaattccagctccaatagcgtatatttaagttgttgcagttaaaaagctcgtagttggatttcgggtgggcgccgccggtctcccattgggtatgtactggcgtggcccgccttgctgtcggggacgctctcccgggcttcactgctcgggaagcggagtcgacgacgttactttgagtaaattagagtgttcaaagcaagccatcgctctgaatatgatagcatgggataacacgacaggactctggcctatcgtgttggtctgtaggaccggagtaatgattaagagggacagtcgggggcattcgtattccgttgtcagaggtgaaattcttggatttacggaagacgaacatctgcgaaagcatttgccaaggatgttttcattgatcaagaacgaaagttgggggctcaaagacgattagataccgtcgtagtctcaaccataaacgatgccgactagggattggcggacgttctttcgatgactccgccagcacctcatgagaaatcaaagtttttgggttccgggggagtatggtcgcaaggctgaaacttaaaggaattgacggaagggcaccaccaggcgtggagcctgcggcttaatttgactcaacacgggaaaacttaccaggtccagacataggaaggattgacagattgagagctctttcttgattctatgggtggtggtgcatggccgttcttagttggtgggttgccttgtcaggttgattccggtaacgaacgagacctcagcctgctaaatagcgacgtctgcctgtgcagtcggcccgcttcttagagggactgttggcgtctagccaatggaagtttgaggcaataacaggtctgtgatgcccttagatgttctgggccgcacgcgcgctacactgatacgttcagcgagcccctgctccgaaaggtgtgggtaatcttgcaaaccgtatcgtgatggggatagaacattgcaattattgttcttcaacgaggaatgcctagtaagcgcgagtcatcaactcgcgttgattacgtccctgccctttgtacacaccgcccgtcgctcctaccgattgaacgtgctggtgaagcgttaggactggagcccggctctaggttctcctggtccgggacccgggaatttcgttgaaccctcccgtttagaggaaggagaagtcgtaacaaggtttccgtaggtgaacctgcggaaggatcattgaatccatcccctgcccccccagcgaaccgtcccagtacccctgtcgggc

SEQ ID NO: 3 - RNA, 1845 nt, unidentified
  source 1..1845: mol_type = other RNA; note = Sphaerica sp.
ccatgcatgtctaagtataagctgattatactgtgaaactgcgaatggctcattaaatcagttagagtttatttgatggtaccttactactcggataaccgtagtaaagctacagctaatacgtgcgcaaatcccgactcacgaagggacgtatttattagattcaagaccgaccgtgctcccgcacgtccttggtgaatcatggtaacttcacgaatcgcatggcctcgtgccggcgatgtttcattcaactttctgccctatcaactttcgacggtagtatagaggactaccgtggtagtaacgggtgacggaggattagggttcgattccggagagggagcctgagaaacggctaccacatccaaggaaggcagcaggcgcgcaaattacccaatcctgacacagggaggtagtgacaataaatatcatgcgatggccaatcggtcgtcgactggaatgagtacagtttaaatcccttaacgaggatccattggagggcaagtctggtgccagcagccgcggtaattccagctccaatagcgtatatttaagttgttgcagttaaaagctcgtagttggatttcgggtgggcgccgccggtctcccattgggtatgtactggcgtggcccgccttgctgtcggggacgctctcccgggcttcactgctcgggaagcggagtcgacgacgttactttgagtaaattagagtgttcaaagcaagccatcgctctgaatatgatagcatgggataacacgacaggactctggcctatcgtgttggtctgtaggaccggagtaatgattaagagggacagtcgggggcattcgtattccgttgtcagaggtgaaattcttggatttacggaagacgaacatctgcgaaagcatttgccaaggatgttttcattgatcaagaacgaaagttgggggctcaaagacgattagataccgtcgtagtctcaaccataaacgatgccgactagggattggcggacgttctttcgatgactccgccagcacctcatgagaaatcaaagtttttgggttccggggggagtatggtcgcaaggctgaaacttaaaggaattgacggaagggcaccaccaggcgtggagcctgcggcttaatttgactcaacacgggaaaacttaccaggtccagacataggaaggattgacagattgagagctctttcttgattctatgggtggtggtgcatggccgttcttagttggtgggttgccttgtcaggttgattccggtaacgaacgagacctcagcctgctaaatagcgacgtctgcctgtgcagtcggcccgcttcttagagggactgttggcgtctagccaatggaagtttgaggcaataacaggtctgtgatgcccttagatgttctgggccgcacgcgcgctacactgatacgttcagcgagcccctgctccgaaaggtgtgggtaatcttgcaaaccgtatcgtgatggggatagaacattgcaattattgttcttcaacgaggaatgcctagtaagcgcgagtcatcaactcgcgttgattacgtccctgccctttgtacacaccgcccgtcgctcctaccgattgaacgtgctggtgaagcgttaggactggagcccggctctaggttctcctggtccgggacccgggaatttcgttgaaccctccgtttagaggaaggagaagtcgtaacaaggtttccgtaggtgaacctgcggaaggatcattgaatccatccctgcccacccagcgaaccgtcccagtacccctgtcggcgcccttggcggggcgcccgccagcggccgggccgtcgcccagtacgggcggcgcc

(19) United States
(12) Patent Application Publication
(10) Pub. No.: US 2026/0209683 A1
(43) Pub. Date: Jul. 23, 2026

(54) LIPID PRODUCING MARINE MICROALGA

(71) Applicant: HUTANBIO LTD., Cambridge (GB)

(72) Inventors: John A.C. ARCHER, Cambridge (GB); Noor Azlin MOKHTAR, Nilai (MY)

(21) Appl. No.: 19/140,475

(22) PCT Filed: Dec. 14, 2023
(86) PCT No.: PCT/EP2023/085959

§ 371 Date: Jun. 18, 2025

(30) Foreign Application Priority Data
Dec. 19, 2022 (MY) PI2022007223

(51) Int. Cl. C12N 1/125; B01D 53/85; C10L 1/02; C12P 7/6427; C12P 7/6431; C12P 7/6432; C12P 7/649; C12R 1/89

(52) U.S. Cl. CPC C12N 1/125; B01D 53/85; C10L 1/02; C12P 7/6427; C12P 7/6431; C12P 7/6432; C12P 7/649; B01D 2251/95; C10L 2200/0476; C12R 2001/89

(57) ABSTRACT

The invention provides marine microalgal strain of the Ulvales order of the Ulvophyceae class which have utility in the efficient and environmentally beneficial preparation of triacylglycerides and polyunsaturated fatty acids, which can in turn be used for fuels or feeds.

### CROSS-REFERENCE TO RELATED APPLICATIONS

This patent application claims the benefit of priority of Malaysian patent application PI2022007223 filed 19 Dec. 2022 and which is herein incorporated in its entirety.

### TECHNICAL FIELD

The present invention relates generally to novel marine microalgae and novel methods and materials relating to the same, for example to the use of microalgae to produce fuels, speciality chemicals or other products.

### BACKGROUND TO THE INVENTION

Strategies to limit global warming to 2° C. above pre-industrial temperatures center on capping future CO2 emission to levels below that of current fossil fuel reserves. Were these options to be adopted, a significant proportion of fossil fuel reserves will remain unused in the period 2020-2050 and although the energy and petrochemical sector is likely to remain the predominant supplier in the coming period, alternative and sustainable sources of petrochemicals commodities are nevertheless urgently required.

Transport generates approximately 29% of global CO2 emissions. Because they require high energy-density liquid fuels, the most challenging transport modes to decarbonise are marine, heavy transport and aviation. These sectors, particularly marine and heavy transport, are essential to the smooth operation of the world economy.

Biofuels such as bioethanol produced by fermentation of biomass derived sugars, or fatty acid methyl ester biodiesel produced by transesterification of biologically produced triacylglycerides, are a class of high-energy hydrocarbons that are synthesised by cells. Photosynthetic organisms are the most energy efficient source of these molecules.

The microalgal cell is the most important component of the algal biofuel production system. Its photosynthetic and metabolic capabilities define the operational limits of biomass and triacylglyceride synthesis which controls biofuel productivity. Microalgae are tremendously diverse and have expanded to occupy every terrestrial, freshwater and marine environment capable of supporting photoautotrophic metabolism [1]. Despite, however, the huge microalgal biodiversity available, which is estimated to number a million species [2,3], screening for biofuel producing strains has been limited in scale and largely confined to pre-existing academic culture collections which number less than 50,000 strains [4,5,6,7]. Performance testing in large scale biofuel programs has therefore been limited to Chlorella, Desmodesmus, Monoraphidium, Scenedesmus and Nannochloropsis strains which respond poorly to abiotic and biotic stresses leading to very low biomass production and lipid content [8,9].

As explained above, new strains with higher real-world triacylglyceride productivity are required that can operate efficiently under the multiple abiotic stresses experienced during culture.

US2014/0199739 (Calleja et al) relates to strains of microalgae belonging to the Isochrysis genus, which are reported to produce polyunsaturated fatty acids, notably eicosapentaenoic acid, in mixotrophic mode. This publication further relates to methods for selecting and culturing such strains, using a discontinuous supply of light in the form of flashes. This is intended to permit possible savings in space and energy related to the supply of a weaker light intensity.

U.S. Pat. No. 8,067,225 (Fernandez Sevilla et al.) relates to a strain of Scenedesmus microalga species and to the use thereof for animal and/or human consumption and in the production of carotenoids. The strain was isolated at the Las Palmerillas experimental station, Cajamar.

WO2005003309 relates to the culture of isolated pseudopterosin-producing alga of the clade B genus Symbiodinium. Preferred conditions are a 14-10 hour light-dark cycle, and illumination of about 30 up to 150 μmol photons m⁻²s⁻¹ at a temperature from about 20° C. to about 30° C.

US2015/0337255 relates to a culture method for microalgae that cultures unicellular green microalgae belonging to the genus Coccomyxa and groups of organisms closely related thereto, or the Watanabea clade, in an open outdoor culture system using broth having a pH of 4 or lower. It further relates to a culture method for microalgae of the genus Pseudococcomyxa.

It can be seen that the provision of novel microalga species, particularly those with novel adaptations to the environment, would provide a contribution to the art.

### SUMMARY OF THE INVENTION

We carried out a bioprospecting survey of the Malacca Straits and South China Sea. Both are distinguished by very high rates of solar radiation, year-round high temperatures with variable/high salinity seawaters (solar radiation up to 2100 μM photons·s⁻¹·m⁻²; year-round high seawater temperatures 28° C.-30° C.; variable high salinity waters 32-34 PSU). These conditions can provide the optimal marine environment for the evolution of high-light, high-temperature, high-salinity adapted microalgae.

We have developed an efficient and scalable fourth generation marine microalgal biofuel production technology consisting of a suite of multiple isolates of a completely new genus of extremophile marine microalgae we have named Sphaerica. These isolates have been engineered using directed-evolution approaches to create highly efficient carbon capture and conversion production strains which can be cultured in large format photobioreactors using atmospheric, compressed, recovered CO₂ or industrial flue gas and combined with appropriate biomass/lipid downstream processing steps to produce useful product classes including carbon-neutral biofuels based on or derived from Sphaerica triacylglycerides (“bio-oil”), polyunsaturated fatty acids (PUFA) and high protein, PUFA enriched, fish and animal feed. In addition, Sphaerica can act as effective, sustainable and economic carbon capture decarbonisation agent for industrial flue gases.

In particular, examples are provided of directed evolution engineered Sphaerica strains which can utilise high levels of sunlight, temperature and high variable salinity. Therefore, they are capable of highly efficient generation of biomass, bio-oil, PUFA and other biomass-derived products in Tropical and Sub-tropical regions where its growth requirements (light, temperature and salinity) are met. In addition, Sphaerica can also be cultured in artificial systems such as using man-made light and heat sources in locations outwith the sub-Tropical/Tropical zone.

Three Sphaerica isolates HB001, HB002 and HB003 have been deposited with the Culture Collection of Algae and Protozoa. In addition, 19 other Sphaerica spp. have been isolated and characterised. Collectively these represent part of a distinct new genus of unicellular marine microalgal defined by phenotypic and genotypic traits described herein.

Thus in one aspect, the invention provides novel marine microalgal strain of the Ulvales order of the Ulvophyceae class characterised by a novel combination of phenotypic and genotypic properties.

Such algal strains can be provided, in the light of the present disclosure, by screening of microalgae or microalgal biofilms from marine environments, for example by using media described herein comprising filtered seawater supplemented with vitamin-free nitrogen sources, phosphorous and trace elements.

In another aspect there is provided a process for making derived (mutant) strains, for example from parent deposited strains or screened strains.

The invention further provides, in relation to marine microalga of the invention, methods of culturing, storing, or using said strains e.g. to produce biomass, or produce products.

In another aspect there is provided a method of producing microalgal biomass or producing a desired product produced by microalgal culture, which method comprises culturing a population of marine microalgae of the invention.

Populations of microalgae will collectively have the characteristics described above e.g. an average EPS thickness of around 280 nm etc.

Examples of products include biofuel or biofuel additives comprising microalgal triacylglycerides, polyunsaturated fatty acids (PUFA) and high protein foods or feeds. Additionally the microalgal capsules which are sloughed off after division can be a rich source of sucrose which provides a source of low carbon sugar for fermentations.

The invention further provides these products obtained or obtainable from the strain e.g. in isolated or enriched form, or downstream products utilising these microalgal products.

In another aspect there is provided use of as a microalgal strain or related method of the invention for carbon capture from a CO2 containing gas, which is optionally an industrial flue gas.

Some of these aspects and embodiments will now be described in more detail.

### DETAILED DESCRIPTION OF THE INVENTION

The present invention relates to novel marine microalgal strains of the Ulvales order of the Ulvophyceae class. This new Chlorophyte genus is termed Sphaerica herein.

In support of this invention, three Sphaerica isolates HB001, HB002 and HB003 have been deposited 26 Apr. 2022 with Culture Collection of Algae and Protozoa as patent strains with the following codes:
CCAP 2271/1 Sphaerica sp. HB001
CCAP 2271/2 Sphaerica sp. HB002
CCAP 2271/3 Sphaerica sp. HB003

Culture Collection of Algae and Protozoa, Scottish Association for Marine Science (SAMS) Ltd. Oban, Argyll PA37 1QA, UK

These deposits are incorporated herein by reference. The isolated strains of the present invention have not previously been made available to the public.

As used herein, HB002 may also be referred to as “HBMSM0012”. HB003 may be referred to as “HBMSM0031”. HB001 may be referred to as “HBMSM0061”.

In addition to HB001, HB002, and HB003, nineteen other Sphaerica spp. have been isolated and characterised by the present inventors: HBMSM0011; HBMSM0013; HBMSM0014; HBMSM0015; HBMSM0021; HBMSM0022; HBMSM0023; HBMSM0032; HBMSM0033; HBMSM0034; HBMSM0051; HBMSM0053; HBMSM0054; HBMSM0062; HBMSM0063; HBMSM0064; HBMSM0065; HBMSM0081 and HBMSM0082.

Collectively these deposited strains represent part of a distinct new genus of unicellular marine microalgal defined by the advantageous multigenic traits described herein. Small subunit 18S rRNA sequence data prove the strains are of independent and genetically heterogenous origin (FIG. 6). More specifically, the full length HB001, HB002 and HB003 18S rRNA sequences were used as initial inputs for phylogenetic the analysis that generated FIG. 6. The Sphaerica HB001, HB002 and HB003 18S rRNA sequences are 1797 bp long and were derived from functional annotation of the Sphaerica genome sequences of each HB species, in particular by sequence conservation across closely related genera (Chlorophyta defined by Lin, G. M., Lai, Y. H., Audira, G., &; Hsiao, C. der. (2017). A Simple Method to Decode the Complete 18-5.8-28S rRNA Repeated Units of Green Algae by Genome Skimming. International Journal of Molecular Sciences, 18 (11). https://doi.org/10.3390/IJMS18112341.

For completeness it is noted that sequences from two public database sequence entries termed AB058352 and AB058374 are included in FIG. 6. These sequences were 1746 bp in length and were apparently generated using oligonucleotide primers that bind highly conserved regions internal to the full length 18S rRNA gene from certain source strains. However, as discussed in more detail in Example 3 below, the source organisms for these partial sequences were seemingly not deposited and have proved to be unavailable from any other source. Nor is any further information concerning complete sequence, or any phenotypic information (for example structural, ultrastructural and metabolic etc) available. Therefore, the organisms from which these sequences were apparently obtained remain obscure.

Thus in one aspect of the invention there is provided a marine microalgal strain characterised by at least 5, 6, 7, or 8 of the following properties (i)-(viii):
(i) being unicellular and uni-nucleate;
(ii) non motile, without cilia nor flagellae;
(iii) having generally spherical cells;
(iv) having a diameter of about 1.00 to 11.0 μm, more preferably 1.5-10.3 μm;
(v) having an extracellular polysaccharide capsule 0.180 μm to 0.400 μm thick, more preferably 0.210 to 0.350 μm thick;
(vi) having chloroplasts that are parietal with a single pyrenoid;
(vii) includes lipid bodies;
(viii) capable of asexual reproduction within maternal capsule through one into 2 nonmotile daughter cells or through two sequential divisions at approximately 90° to each other into 4 nonmotile daughter cells.
said strain also:
(ix) having from 120 to 300 thylakoid membranes per chloroplast; and
(x) having an 18S rRNA which exhibits 97% or higher base sequence homology with the 18S rRNA sequence shown in the FIG. 5.

In one embodiment the strain is characterised by having all of features (i)-(x).

In one embodiment the strain is characterised by:
(i) the chloroplasts in mature cells (optionally exhibiting penta-radial symmetry) and are parietal occupying upper cell hemisphere with lobes extending into the remaining hemisphere which contains the lipid bodies; and/or
(ii) the thylakoids enclose an approximately 17 nm wide lumen and form a continuous 40 structure throughout the chloroplast with thylakoids running parallel to the cell membrane; and/or
(iii) the neutral lipid content can make from 5% to 70%, optionally 5% to 65%, of cell dry mass.

45 In one embodiment the strain is selected from HBMSM0011; HBMSM0013; HBMSM0014; HBMSM0015; HBMSM0021; HBMSM0022; HBMSM0023; HBMSM0032; HBMSM0033; HBMSM0034; HBMSM0051; HBMSM0053; HBMSM0054; HBMSM0062; HBMSM0063; HBMSM0064; HBMSM0065; HBMSM0081 and HBMSM0082.

In one aspect of the invention provides a microalgal strain characterized in that it consists of an isolated strain deposited at the CCAP under accession number CCAP 2271/1, CCAP 2271/2 or CCAP 2271/3.

In one aspect of the invention provides a microalgal strain characterized in that the strain is a Sphaerica strain and is present in the same phylogenetic clade within the Ulvophyceae as a strain deposited at the CCAP under accession number CCAP 2271/1, CCAP 2271/2 or CCAP 2271/3 (see FIG. 6. As noted above CCAP 2271/1 is Sphaerica sp. HB001 aka HBMSM0061/20; CCAP 2271/2 is Sphaerica sp. HB002 aka HBMSM0012/20; CCAP 2271/3 is Sphaerica sp. HB003 aka HBMSM0031/20).

As explained herein, microalgal strains of the invention may be obtained or obtainable by screening of microalgae, optionally microalgal biofilm, from a marine environment using filtered seawater supplemented with vitamin-free nitrogen sources, phosphorous and trace elements.

For example, Sphaerica HB001, HB002 and HB003 strains are capable of rapid propagation to high biomass content on simple media comprising filtered non-sterile seawater 38-45 PSU supplemented with FSM medium (NaNO₃ 7.0592 mM, NaH₂PO₄·H₂O 0.3261 mM, FeCl₃·6H₂O 0.0233 mM, C₁₀H₁₄N₂Na₂O₈·2H₂O 0.0259 mM, CuSO₄·5H₂O 7.85004 10⁻⁵ mM, ZnSO₄·7H2O 0.000152 mM, CoCl₂·6H₂O 0.000154 mM, MnCl₂·4H₂O 0.001818 mM, Na₂MoO₄·2H₂O 5.20730 10⁻⁵ mM.); without vitamin supplements nor antibiotics nor antifungals (see FIG. 7).

Screening of microalgae from a marine environment can thus be achieved using a selection medium such as “FSM” by:
(i) culturing a diversified pool of microalgae in any of said the media;
(ii) maintaining said culture over several generations;
(iii) isolating the strain(s) for which the number of cells has most increased during said generations.

Culture conditions may include:
(i) a culture temperature of between 18° C. and 40° C. e.g. 30° C.-35° C.
(ii) an illumination of 500 μM photons PAR 400 nm-700 nm·s⁻¹·m⁻² or higher

The strains of the invention demonstrate excellent properties.

For example Sphaerica HB001, HB002 and HB003 show prolonged biomass growth curve in air lift flat panel photobioreactor in which cell biomass increased from approximately 1.8 kg·m⁻³ cell dry weight to approximately 7.2 kg·m⁻³ cell dry weight over 50 days continuous growth (FIG. 7). These strains are capable of rapid propagation to high biomass content under high light levels up to 2100 μM photons·s⁻¹·m⁻² solar insolation on simple media comprising coarse-filtered (e.g. 5 μm) sea water supplemented with nitrogen, phosphorous and trace elements. This is shown, for example, in the biomass growth curve achieved in the photobioreactor at up to 2100 μM photons·s⁻¹·m⁻² sunlight 12 hours day/12 hour night (FIG. 5). There is no requirement to sterilise the nitrogen source, thereby avoiding the energy requirement for such sterilisation.

Furthermore Sphaerica strains are capable of rapid propagation to high biomass content without cooling under variable high temperatures on the same simple media. This is shown, for example, in the biomass growth curve in FIG. 8 where the culture temperatures varied from 24° C.-35° C.

We have shown that Sphaerica will remain viable at 45° C. for 72 hours. While it does not grow rapidly at 45° C., it remains viable and quickly restores rapid cell growth when the temperature drops below 40° C. Furthermore it remains metabolically active down to at least 4° C.

Thus in one embodiment the microalgal strain is characterised by:
(i) having higher growth rates when cultured externally on a 12:12 day/night cycle having a maximum daytime insolation of greater than or equal to about 1600, 1700, 1800, 1900, 2000, 2100 μM photons s⁻¹·m⁻² as compared to a maximum of 150, 200, 250, or 300 μM photons s⁻¹·m⁻²;
(ii) remaining viable after 72-hours culture at 45° C.

As noted above Sphaerica strains are capable of rapid propagation to high biomass with very high lipid content (>60% cell dry weight lipid) under nitrogen limitation conditions on simple media (see FIG. 8). The strains can produce simple short chain triacylglycerides (“TAG”-C₁₆-C₁₈) as well as polyunsaturated fatty acids (“PUFA”). This can be seen, for example, in the Sphaerica HB003 GC-MS plot of total lipid extract (FIG. 10) and FIG. 14 which shows high levels of biofuel TAG components hexadecanoic acid, octadecadienoic acid, and high value PUFA derivatives from eicosadienoic acid, arachidonic acid, linolenic acid and others.

In one aspect there is provided a process for making a derivative strain of any of the strains of the invention described above, for example by directed evolution.

Typically the derived strains possess biological activities that are the same or similar (or improved) compared to those of the deposited strains i.e. typically the derived strains will maintain or improve the key favourable characteristics of the strains described herein.

Also provided processes for making a derived strain from the deposited strains by mutagenesis & selection.

There are also provided processes for making a derived strain from deposited strain by gene insertion\deletion\modification.

The derived strains (which are “mutants”) are those which have undergone a mutation, i.e. a change in a nucleic acid sequence (such as a gene sequence) or amino acid sequence, for example as compared to a nucleic acid or amino acid sequence present in an original parental organism, for example a deposited strain of the invention.

Mutations can occur spontaneously, or can be introduced, for example using molecular biology methods. In particular examples, a mutation includes one or more nucleotide substitutions, deletions, insertions, or combinations thereof.

In one embodiment directed evolution comprises one or more of the following steps:
(i) culturing the deposited parent strain under an environmental stress selected from: high salinity and/or high insolation, wherein the intensity of said stress is increased step-wise;
(ii) maintaining said culture over several generations;
(iii) isolating the strain(s) for which the number of cells has most increased during said generations.

Also provided is a process for making a derived strain, which process comprises mutagenesis and selection of one of the strains of the invention.

To select or screen the strains, various strains may be cultured, in parallel, on microplates, in the same enclosure, with a precise monitoring of the conditions and of the development of the various cultures.

Methods of passive or active mutagenesis and selection may generally comprises one or more of the following steps:
(i) culturing the deposited parent strain under conditions promoting mutagenesis;
(ii) maintaining said culture over several generations;
(iii) isolating the strain(s) for which the number of cells has most increased during said generations.

In addition to those methods, DNA mutagenesis and screening for mutants may be utilized. DNA mutagenesis is well known to these skilled in the art and includes a variety of chemical and enzymatic methods.

Examples of such methods include but are not limited to exposure of the subject organism to ultraviolet light (UV), nitrous acid, N-methyl-N′-nitro-N-nitrosoguanidine (NG), and 4-nitroquinoline-N-oxide (4NQO) (Leninger (1972) Biochemistry. Worth Publishers Inc., NY; Jeffrey H. Miller (1972) “Experiments in Molecular Genetics. Cold Spring Harbor Laboratory, Cold Springs Harbor, New York).

By way of example, mutants may be produced by exposure of the Sphaerica cells to UV light for a period of about 10 to about 180 seconds, preferably 45 to about 90 seconds, and most preferably 65 to 75 seconds. Mutagenesis involving NG may involve varying the concentration of the NG and exposure time. By way of example NG, at about 13 milligrams/liter (mg/L) to about 400 mg/L of NG may be used for a period of exposure of about 15 minutes to about 120 minutes.

As used herein, the term “genetically modified” refers to any change in the endogenous genome of a wild type cell or to the addition of non-endogenous genetic code to a wild type cell, e.g., the introduction of a heterologous gene. More specifically, such changes are made by the hand of man through the use of recombinant nucleic acid technology or mutagenesis. The changes can involve protein coding sequences or non-protein coding sequences such as regulatory sequences as promoters or enhancers or other cis- or trans-acting regulatory DNA sites or RNA species.

Generally speaking, those skilled in the art are well able to construct vectors and design protocols for recombinant gene expression or more modification of target genomes. Suitable vectors can be chosen or constructed, containing appropriate regulatory sequences, including promoter sequences, terminator fragments, polyadenylation sequences, enhancer sequences, marker genes and other sequences as appropriate. Examples of mutagenesis techniques include, but are not limited to, oligonucleotide directed mutagenesis, linker scanning mutations or oligonucleotide directed mutagenesis using polymerase chain reaction. For further details see, for example, Molecular Cloning: a Laboratory Manual: 2nd edition, Sambrook et al, 1989, Cold Spring Harbor Laboratory Press or Current Protocols in Molecular Biology, Second Edition, Ausubel et al. eds., John Wiley & Sons, 1992.

Publication WO2014/076571 describes specific methods for modifying genetic material in algal cells that includes the use of rare-cutting endonucleases (e.g. a homing endonuclease or a TALE-Nuclease) to modify said target genome sequences.

Other methods of modification are well known to those skilled in the art

The clustered regularly interspaced short palindromic repeat (CRISPR)/CRISPR associated protein (Cas) system is an adaptive immune mechanism found in bacterial and archaeal species that allows the host to combat pathogens, such as bacteriophages (Barrangou, R. et al. Science 315, 1709-1712 (2007); Marraffini, L. A. & Sontheimer, E. J. Science 322, 1843-1845 (2008); Bhaya, D., Davison, M. & Barrangou, R. Annual review of genetics 45, 273-297 (2011); Garneau, J. E. et al. Nature 468, 67-71 (2010)). Bacteriophage-derived 30-bp DNA fragments are inserted into the CRISPR locus of the host cell and transcribed as CRISPR RNAs (crRNAs). These form a complex with trans-encoded RNA (tracrRNA) and CRISPR-associated (Cas) proteins, and the complex introduces site-specific cleavage at DNA sites that match the sequence of the crRNAs.

CRISPR-Cas9 is a type II CRISPR-Cas system. CRISPR-Cas9 system from Streptococcus pyogenes is used in the art as a simple and versatile tool for RNA guided genome editing (RGE) in different organisms. In Cas9 mediated RGE, a single or duplex short RNA molecule (guide RNA or gRNA) directs Cas9 to target the desired DNA site for genome modification or transcriptional control. gRNA-Cas9 recognizes targeted DNA by gRNA-DNA pairing between 5′-end leading sequence of gRNA (referred as gRNA spacer) and one DNA strand (complementary stand of protospacer). Cas9 also requires the presence of protospacer-adjacent motif (PAM) in the target site following the gRNA-DNA pairing region.

The approximate 20 nt long gRNA spacer sequence could be readily programmed to target DNA sites with PAM. But gRNA-Cas9 also recognizes PAM sites that match gRNA spacer imperfectly, resulting off-target risk in genome editing. As a result, designing gRNA with highly specific spacer sequence is critical for RGE.

CRISPR-cas9 plasmids for use in plants are commercially available, for example from Addgene—see: www.addgene.org/crispr/plant/

In the context of the present inventions, for example, the genome sequence of HB003 has been determined by the inventors and from these data, genes of the microalgae of the invention identified in its genome sequence may be targets for editing using CRISPR-cas9 plasmids (i.e. be used to provide “gRNAs”) or other RNA-guided site-specific modification techniques, thereby modifying those genes in order to modify the phenotypes the strains.

It further provides marine microalgae which are derived from of any of these, for example by culture and re-selection, random or directed mutagenesis, or by other gene insertion, deletion, or modification.

It further provides marine microalgae which are obtained or obtainable by screening of microalgae from a marine environment using a selection process as described herein.

Any of these Sphaerica strains can be utilised in the methods of the invention and may be referred to herein for brevity as a “marine microalga of the invention”.

Such marine microalgae of the invention may be characterised by any of the properties referred to herein

For example the (derivative) microalgal strain may be a directed evolution engineered derivative strain like Sphaerica HB001, HB002 and HB003. Such strains are capable of rapid propagation to high biomass content under variable high salinity conditions on simple media comprising filtered non-sterile sea water supplemented with nitrogen, phosphorous and trace elements. By way of example, FIG. 9 shows these strains propagated efficiently as culture salinity rose from 40 PSU to 50 PSU from approximately 1.8 kg·m⁻³ to 7.2 kg·m⁻³.

Thus in one embodiment the strain is capable of more efficient growth in vitamin-free media having a PSU of greater than or equal to 45 PSU, as compared to otherwise identical media with 3 PSU.

In one embodiment of the invention there is provided a biologically pure culture of the strains discussed herein e.g. a slope culture or culture in a liquid medium or broth.

Strains may be harvested using conventional washing, filtering or sedimentary techniques such as centrifugation, or may be harvested using a cyclone system. Harvested cells can be used immediately or stored as described herein. A non-limiting example of freeze-drying techniques suitable for microorganisms is described in WO 2008041786 A1.

In one embodiment of the invention there is provided a freeze-dried sample, a liquid nitrogen-frozen sample, or a frozen preparation in glycerol, of any of said strains.

In one embodiment of the invention there is provided a cell extract; a cell suspension; a cell homogenate; a cell lysate; or a cell pellet of any of said strains.

In one embodiment of the invention there is provided a culture broth of, or a cell free or substantially cell free culture broth of, any of said strains.

In another aspect there is provided a method of producing microalgal biomass or producing a desired product or compound produced by microalgal culture, which method comprises culturing a population of marine microalgae of the invention.

Therefore the cultured marine microalgae (e.g. as biomass) may be the product per se, or the method may be used to produce a desired compound, such as a chemical or biochemical product of commercial or industrial interest, wherein the compound is recovered from the culture.

The culture system may comprise one or more detectors for detecting one or more parameters selected from: pH, CO₂, salinity and light intensity.

In one embodiment said marine microalgae are cultured:
(i) at a temperature of between about 20° C. to about 50° C., preferably 28° C.-35° C.; and/or
(ii) at a pH of between 6 and 11, preferably about pH 8; and/or
(iii) a salinity of between 15 to 63 PSU, preferably 30 to 40 PSU; and/or
(iv) a photonic intensity of up to 2100 μmol photons m⁻²·s⁻¹ 400 nm-700 nm Photosynthetically Available Radiation.

In one embodiment said marine microalgae are cultured in a culture medium, which culture medium is characterised by a salinity range 20-45 PSU, e.g. about 20, 25, 30, 35, 38, 40, 42 or 45 PSU.

Those skilled in the art will understand that salinity can be adjusted, in accordance with the present disclosure, according to economical factors relevant to local water resources. For example 20 PSU is brackish water, and Pacific/Atlantic/Indian Oceans/Great Australian Bight, Bass Straits, Tasman Sea, Coral Sea, Arajura Sea or any other waters or seas that are 30-36 PSU.

As exemplified below, Sphaerica HB001, HB002 and HB003 strains when cultured on simple media can efficiently utilises industrial flue gas (agricultural waste; coconut husks; palm oil empty fruit bunch) as a direct source of CO2-see e.g. the HB003 biomass growth curve (FIG. 11).

Thus in one embodiment the marine microalgal cells are mixed during culture via air bubbling into the culture media and/or are supplemented with CO2, which is optionally industrial flue gas.

For use in the present invention, industrial/agricultural flue gas may be captured from source (e.g. the chimney), cooled passively or actively, and preferably compressed for supply to the culture (e.g. in a PBR). Such flue gas may include a relatively high particulate component and high levels (e.g. about 15%) CO2 by volume.

In one embodiment an additional source of CO₂ is provided e.g. 0.1 to 15%, or 0.1 to 10% or 5%.

Sphaerica isolates have been shown to start photoautotrophic growth as soon as day starts, so can be grown at very low photonic fluxes (e.g. from 1 μM photons·s-1·m-2). However, productivity and CO₂ capture and conversion into biomass and triacylglycerides by Sphaerica is be increased by increasing photon flux using artificial light sources to provide continuous illumination (see e.g. HB003 biomass growth curve (FIG. 11). Under high photon flux, e.g. >500 μM photons·s-1·m-2 when most other microalgal strains are photo-inhibited, Sphaerica remains photosynthetically active by dint of its large number of thylakoid membranes acting to attenuate photon flux by absorbing excess photons in outer thylakoid layers and radiating the energy as heat, thus reducing the available photon flux in more interior thylakoid layers. Therefore they can grow at high photon fluxes, unlike certain current algal biofuel strains that exhibit strong photoinhibition above 500 μM photons·s-1·m-2.

For industrial applications a preferred range is the 100 μM-2100 μM photons·s-1·m-2. In one embodiment the marine microalgae are cultured under artificial light.

In one embodiment said marine microalgae are cultured at a photonic intensity of between about 20 and about 2100 μmol photons m⁻²·s⁻¹ Photosynthetically Available Radiation (400-700 nm).

Culturing is preferably under ambient external light (i.e. under a natural day/night cycle) whereby the culturing is in a location providing between 100 and 2100, e.g. about 100, 200, 300, 400, 500, 600, 700, 800, 900, 1000, 1100, 1200, 1300, 1400, 1500, more 1600, 1700, 1800, 1900, 2000, or 2100 μmol photons m⁻²·s⁻¹ at normal peak daytime exposure.

In another embodiment the marine microalgae are cultured externally under natural day/night cycle, and wherein:
(i) the maximum light intensity in the peak day part of the cycle is at least or about 1000, 1100, 1200, 1300, 1400, 1500, 1600, 1700, 1800, 1900, 2000, 2100 or 2200-μmol photons m⁻²·s⁻¹ at peak daytime exposure; and/or
(ii) the maximum temperature at the peak day part of the cycle is between 30° C. to about 50° C., optionally at least 35° C., 38° C., 48° C.; and/or
(ii) the maximum salinity during culture is at least 30, 35, 40, 45, 50, 55, 60 or 63 PSU.

In one embodiment said marine microalgae are cultured at a temperature of between about 20° C. to about 50° C., e.g. 24° C. to 48° C., preferably about 35° C.

In one embodiment said marine microalgae are cultured in a culture medium, which culture medium is characterised by a salinity range 20-60 PSU, e.g. about 20, 25, 30, 35, 40, 45, 50, 55 or 60 PSU.

In one embodiment the culture is photoautotrophic.

For industrial applications, significant cost savings accrue by substituting nitrate by urea a common industrial by-product. It is simple to make up, its three-part constituent formulations are added to water/seawater/industrial brine as required.

Preferred medium is a synthetic medium prepared from:
(1a) either a natural source of salt-water e.g. sea water, brackish water, or a salt-lakes, or
(1b) an industrial brine by-product e.g. from desalination for human consumption and irrigation, power plant cooling towers, produced water from oil and natural gas extraction, acid mine or acid rock drainage, reverse osmosis reject, chlor-alkali wastewater treatment, pulp and paper mill effluent, and waste streams from food and beverage processing.
(2) an additional source or sources of a nitrogen containing compound (e.g. nitrate or urea) and phosphate;
(3) optionally an additional source of trace elements; and
(4) preferably no added vitamins to the medium, nor added antibiotic, nor antifungal supplements.

In one embodiment an additional source of trace elements is provided, and optionally comprises one or more of: FeCl₃, CuSO₄, ZnSO₄, CoCl₂, MnCl₂, or Na₂MoO₄.

Preferred medium is based on non-sterile filtered sea water e.g. Mediterranean, Atlantic, Pacific, South China Sea, Malacca Straits, Indian Ocean, Australian Bight, Timor Sea, Arafura Sea, Gulf of Carpentaria, Coral Sea, Tasman Seas.

These waters are highly saline and oligotrophic.

The media can be re-formulated to a range of salinities by changing the salinity by dilution to e.g. utilising 0 PSU (fresh water) to 60 PSU (industrial brine).

In one embodiment the media comprises filtered sea water supplemented with NaNO₃ about 7 mM; NaH₂PO₄·2H₂O about 0.3 mM; C₁₀H₁₄N₂Na₂O₈·2H₂O about 20 μM; FeCl₃·6H₂O about 25 μM; CuSO₄·5H₂O about 29 nM; ZnSO₄·7H₂O about 150 nM; CoCl₂·6H₂O about 150 nM; MnCl₂·4H₂O about 2 μM; Na₂MoO₄·2H₂O about 50 nM.

In one embodiment the method is performed as batch culture.

The culture process may be sustained as desired according to the scale and desired output or product—for example cells may be kept in mid to late logarithmic growth phase, or incubated to stationary phase.

In one embodiment the method is performed as continuous culture.

The method may be performed using simple open ponds. Alternatively, the method is performed in a culture reactor. In one embodiment the method is performed in a vertical or inclined culture reactor adapted to receive natural sunlight.

Preferably the culture reactor is a photobioreactor (“PBR”) e.g. an air lift column or flat panel.

“PBRs” are artificial constructs in which the light path through the algal culture is optimized for the available incident light. The light path can typically be increased or decreased by changing the dimensions of the photobioreactor tube (in the case of air-lift PBRs) or depth of panel (in the case of flat panel PBRs). The present invention is particularly suitable for practising in PBRs.

In one embodiment wherein the method is for producing desired compounds or compositions selected from:
(i) a biofuel comprising microalgal triacylglycerides; and/or
(ii) polyunsaturated fatty acids (PUFA) and; and/or
(iii) other lipid fractions
(iv) high protein, PUFA rich food or fish- or animal-feed.
(v) sucrose for fermentations.

In one embodiment the culturing methods further comprise the step of recovering, isolating, purifying, or enriching the desired product from the culture.

Sphaerica intracellular metabolites can be purified from cultured cells by a variety of mechanical approaches including bead milling, hydroshearing, pressure treating, hydrothermal liquefaction or sonication. Following cell lysis, Sphaerica bio-oil may be purified by solvent extraction using chloroform/methanol, hexane or other suitable organic solvent system, or by super-critical CO₂.

Methods of purifying peptides or other compounds from heterogenous mixtures are well known in the art (e.g. selective precipitation, proteolysis, ultrafiltration with known molecular weight cut-off filters, ion-exchange chromatography, gel filtration, etc.). A particularly useful technique in this regard is ultracentrifugation (e.g. 150,000×g for over one hour). Further methods which are known to be suitable for protein purification are disclosed in “Methods in Enzymology Vol 182—Guide to Protein Purification” Ed. M P Deutscher, Pub. Academic Press Inc. Typical protocols are also set out in “Protein Purification—principles and practice” Pub. Springer-Verlag, New York Inc (1982), and by Harris & Angal (1989) “Protein purification methods—a practical approach” Pub. O.U.P. UK, or references or subsequent editions thereof.

Where appropriate the product may be derived from the supernatant of the culture, for example after centrifugation, filtration or decanting, followed optionally by precipitation.

In one embodiment the desired product is recovered by tangential flow filtration.

In one embodiment a triacylglyceride composition is recovered from the culture by solvent extraction.

A method of providing a triacylglyceride composition may comprises the steps of:
(a) filtering the cell mass from the culture;
(b) lysing the algal cells in the cell mass in the presence of a polar-solvent, which is optionally methanol;
(c) mixing the polar-solvent and lysed cellular material with a non-polar solvent which is optionally hexane;
(d) recovering the triacylglyceride-containing non-polar solvent layer;
(e) recovered the triacylglyceride from the non-polar solvent layer.

In one embodiment a triacylglyceride composition is recovered from whole or disrupted dried cells by supercritical CO2.

A method of providing a triacylglyceride composition may comprise the steps of:
(a) filtering the cell mass from the culture;
(b) drying the cell mass;
(c) optionally disrupting/lysing cells in the cell mass;
(d) solubilising metabolic products such as triacylglycerides using super-critical CO2 which optionally may also utilise organic co-solvents.

The invention also provides a product obtained or obtainable according to the methods of the invention described herein.

Such a product may, for example, be:
(i) a triacylglyceride enriched composition, or
(ii) a polyunsaturated fatty acids (PUFA) enriched composition.

Typically the compositions will comprise both triacylglyceride and polyunsaturated fatty acids.

A PUFA composition will typically comprise at least linolenic acid; eicosapentaenoic acid; and arachidonic acid.

A triacylglyceride enriched composition may be characterised in that:
(i) the most prevalent fatty acid types in the triacylglyceride are hexadecanoic acid and octadecadienoic acid; and/or
(ii) the composition comprises one, two, three or four of the following compounds: 1-Hexadecyne; cis-13-Octadecenoic acid; trans-13-Octadecenoic acid; 1,4-Eicosadiene; and/or
(iii) the composition does not comprise the following compounds: Tetradecanoic acid; Heptadecanoic acid; Nonadecanoic acid.

The composition, or triacylglyceride enriched composition, may further comprise: Bicyclo[3.1.1]heptane, 2,6,6-trimethyl-, (1.alpha.,2.beta.,5.alpha.); B-Sitosterol; 3,7,11,15-Tetramethyl-2-hexadecen-1-ol.

The triacylglyceride enriched composition may be characterised in that is comprises one or more, or all, of the compounds described in Table 5 hereinafter within the respective % range shown therein.

The triacylglyceride enriched composition may be characterised by a GC-MS profile substantially as shown in FIG. 13 or Table 3, or having a fatty acid composition substantially as shown in Table 2.

The inventors have showed that Sphaerica biofuel can be used as a direct fuel in a fossil diesel:biofuel mixture for diesel internal combustion engines. This direct use avoids the transesterification step conventionally required for diverse and very long chain fatty acids produced from animal and plant sources in typical biodiesel fatty acid methylester fuels. Such conventional methods use 3 molecules of ethanol or methanol per molecule of triacylglyceride; the alcohol being usually sourced from fossil fuels which compromises the biofuel carbon footprint derived from transesterification. Sphaerica triacylglycerides do not require this chemical modification for diesel fuel applications.

Specifically, Sphaerica bio-oil unmodified (neither transesterified nor refined—see below) has been used successfully as a low carbon blend with petroleum diesel as a fuel in a diesel test engine. Preferred blends include 5% bio-oil 95% diesel (B5 EU diesel), B7 7% bio-oil 93% diesel (Asian biofuel standard); B10 10% bio-oil 90% diesel (EU target); B24 24% bio-oil 76% diesel, which is the target for lower carbon marine fuel needed to satisfy IMO2020 regulations.

Employing suitable fuel-handling systems B100 (100% bio-oil) may itself be used as a marine or heavy diesel fuel.

Thus in one embodiment the Sphaerica product is a “drop-in” fuel component e.g. a diesel fuel component, for example for terrestrial automobile or marine applications, or for generator use. One aspect of the invention provides a biodiesel fuel comprising a Sphaerica product as discussed above, optionally 5%, 10%, 24%, or more (e.g. 100%), of said product in combination with the balance of fossil-fuel derived diesel fuel. Such fuel may be used in automobiles, marine engines, or any other application requiring diesel fuel. Those skilled in the art will recognise that the preferred upper limit on the % of “bio-oil” in low carbon fuels will be determined by the capabilities of the diesel injection pump. For example marine engines have sophisticated and powerful injection systems capable of utilising a wide variety of fuels. However, car and truck diesel engines may have limits on the fuels they can use imposed by licensing authorities. Thus the preferred mix will be determined by the relevant application.

Another aspect of the invention provides a fuel product obtained by transesterification of the Sphaerica bio-oil product of, wherein one or more triacylglycerides have been converted to corresponding fatty acid methyl esters. Such transesterification can reduce the viscosity of the fuel product.

Sphaerica triacylglycerides are converted by transesterification (a reaction with excess alcohol, typically methanol, catalysed by KOH to form the corresponding methyl esters and glycerol).

Alternatively the triacylglycerides can be treated by hydrodeoxygenation in the presence of excess hydrogen and a suitable catalyst to alkanes (Choudhary, T. v, & Phillips, C. B. (2011). Renewable fuels via catalytic hydrodeoxygenation. Applied Catalysis A: General, 397, 1-12. https://doi.org/10.1016/j.apcata.2011.02.025)

Another aspect of the invention provides a fuel product obtained by refining the Sphaerica product or Sphaerica cell preparations discussed herein. For example Sphaerica triacylglycerides may be used as a low carbon feedstock either blended or unadulterated may be used in standard petrochemical refining processes, for example as a refinery cracking column feedstock. Those skilled in the art are well aware of standard refining techniques.

Standard petroleum chemical engineering approaches would then derive a range of low carbon petrochemicals such as fuels, solvent, chemicals, additives, paints and plastics.

Another aspect of the invention provides an aviation fuel e.g. generated by simple chemical modification or refinement of a product as discussed herein.

Another aspect of the invention provides a fuel product which comprises a natural Sphaerica triacylglyceride-containing product combined with a trans-esterified or refined product.

Thus non-limiting fuels of the invention include Sphaerica triacylglyceride-containing products blended with petroleum diesel, or blended with Sphaerica triacylglycerides which have been trans-esterified into fatty acid methyl esters, or blended with refined Sphaerica fuel.

The invention may also be used to produce other products—for example cells can also produce the essential human nutrient ω-3 polyunsaturated fatty acids (ω-3 PUFA) for which current production from marine oily fish is unsustainable, and susceptible to heavy metal and radionuclide contamination.

Another aspect of the invention provides a high protein agricultural feedstock comprising microalgal biomass obtained according to the methods of the invention discussed above.

In a further aspect there is provided use of as a microalgal strain of the invention, or a method of the invention, for carbon capture from a CO2 containing gas, which is optionally an industrial flue gas.

### Definitions and General Terminology

“Algae” is an informal term for a large, diverse group of photosynthetic organisms which are not necessarily closely related. Included organisms range from unicellular genera to multicellular forms.

Though popularly considered water plants, algae are not usually grouped in the same kingdom as the so-called true plants, Plantae.

In fact, most algae contain chloroplasts that are similar in structure to cyanobacteria. Algal color is determined by pigments in the chloroplasts. Green chlorophyll is the primary pigment for all algae, but some types have accessory pigments that can mask this color.

Algal species may have discoid or reticulate parietal chloroplasts. “Parietal” means that the chloroplasts lie against the cell walls. “Discoid” means that each cell contains many small chloroplasts.

Algae store energy in the form of carbohydrates—starch or sugars—inside their chloroplasts. Pyrenoids are the starch-storage units. The shape of pyrenoids can vary considerably e.g. spherical, elongated, lenticular, multi-lobed, or compound. Some algal species such as Sphaerica are capable of generating triacylglyceride carbon-energy storage compounds. Triacylglycerides are synthesised in a complex metabolic pathway initiating in the chloroplast and translocated to the endoplasmic reticulum.

“Microalgae” are a large, heterogeneous group of primitive unicellular algae which occur throughout marine or freshwater types of aquatic habitats and moist terrestrial environments (see e.g. Marine Botany, Clinton Dawes, John Wiley & Sons, 1998, pp 168-).

“Homology” (similarity or identity) may be assessed using GAP4 (Staden, R., K. F. Beal, and J. K. Bonfield, The Staden package, 1998. Methods Mol Biol, 2000. 132: p. 30)—see http://staden.sourceforge.net/.

The term “biologically pure culture” or “biologically pure isolate” as used herein refers to a culture of the relevant strain of the invention comprising at least 90%, preferably 95%, preferably 99% and more preferably at least 99.5% cells of the relevant strain.

Measurement of practical salinity unity (“PSU”) is well known in the art, for example using commercially available CTD instruments. Thus seawater of Practical Salinity 35 has a conductivity ratio of unity at 15° C. and 1 atmosphere pressure with a potassium chloride (KCl) solution containing a mass of 32.4356 grams of KCl per kilogram of solution).

Photosynthetically available light (“PAR”) 400-700 nm can be conveniently measured by using commercially available sensors e.g. from LI-COR Biosciences UK Ltd. St. John's Innovation Centre Cowley Road Cambridge CB4 OWS United Kingdom). LICOR provide suitable quantum sensors (for placing on the surface of the photobioreactor) and data loggers.

Sphaerica “bio-oil” is used herein to describe the typically solvent-extracted fuel product made from disrupted or in some means lysed Sphaerica cells containing predominantly triacylglycerides and polyunsaturated fatty acids, and small amounts of other components from Sphaerica cells.

A number of patents and publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

The term “comprising” as used in this specification means “consisting at least in part of”. When interpreting each statement in this specification that includes the term “comprising”, features other than that or those prefaced by the term may also be present. Related terms such as “comprise” and “comprises”, and the terms “including”, “include” and “includes” are to be interpreted in the same manner.

The term “consisting essentially of” when used in this specification refers to the features stated and allows for the presence of other features that do not materially alter the basic characteristics of the features specified.

It must be noted that, as used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

Ranges are often expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

Any sub-titles herein are included for convenience only, and are not to be construed as limiting the disclosure in any way.

The invention will now be further described with reference to the following non-limiting Figures and Examples. Other embodiments of the invention will occur to those skilled in the art in the light of these.

The disclosure of all references cited herein, inasmuch as it may be used by those skilled in the art to carry out the invention, is hereby specifically incorporated herein by cross-reference.

### FIGURES

FIG. 1 to FIG. 3 Sphaerica light microscopy vegetative cell structure

Sphaerica vegetative cells, dividing and daughter cells. Light micrographs ×1000 of Sphaerica sp. HBMSM003 during stages of its cell cycle. Cells were cultured on non-sterile filtered Malacca Straits seawater supplemented with FSM medium (NaNO₃ 7.0592 mM, NaH₂PO₄·H₂O 0.3261 mM, FeCl₃·6H₂O 0.0233 mM, C₁₀H₁₄N₂Na₂O₈·2H₂O 0.0259 mM, CuSO₄·5H₂O 7.85004 10⁻⁵ mM, ZnSO₄·7H₂O 0.000152 mM, CoCl₂·6H₂O 0.000154 mM, MnCl₂·4H₂O 0.001818 mM, Na₂MoO₄·2H₂O 5.20730 10⁻⁵ mM.); without vitamin supplements nor antibiotics nor antifungals and supplied with 15% v/V CO₂ as cooled compressed flue gas. Cultured in outdoor 300 L photobioreactor receiving approximately 12 hours illumination up to 2100 μM photons·s⁻¹·m⁻² 12 hours darkness, 28° C. to 35° C. diurnal temperature range. Cells were visualized unstained under light microscopy ×1000 magnification using an oil-immersion lens and bright field illumination using a Zeiss Axio Imager A2 (Zeiss International Singapore). Images were recorded using Teledyne Photometrics Micropublisher 6 CCD 12.5×10 mm (16 mm diagonal) 6 megapixels Teledyne Photometrics (Shanghai 200032, China).

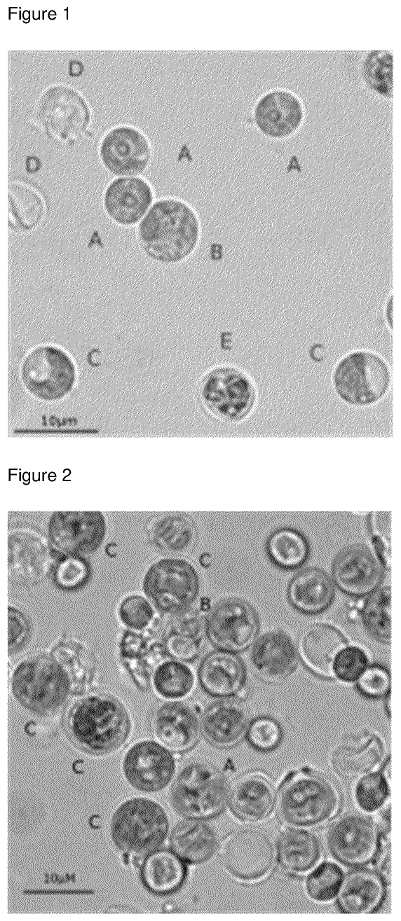

FIG. 1: Bright field image of individual cells showing their characteristic spherical cell shape surrounded by cell capsule. The parietal chloroplast dominates one hemisphere, lipid storage bodies occupy the other. Note absence of cilia and flagellae. Sphaerica is non-motile. Scale bar 10 μM. Key to FIG. 1 (A) apical view of mature Sphaerica cell revealing pyrenoid and parietal chloroplast with penta-radial symmetry; (B) apical view of maternal Sphaerica cell; (C) ventral view of mature Sphaerica cell showing parietal chloroplast and lipid body; (D) ventral view of empty Sphaerica maternal cell capsule; (E) basal view of mature Sphaerica cell.

FIG. 2: Bright field image of individual cells showing developmental stages. Scale bar 10 μM. Key to FIG. 2 (A) maternal cell containing 4 daughter cells each with its own capsule within the intact maternal capsule (B) maternal cell at first division stage (2 daughter cells); (C) maternal cell prior to first division.

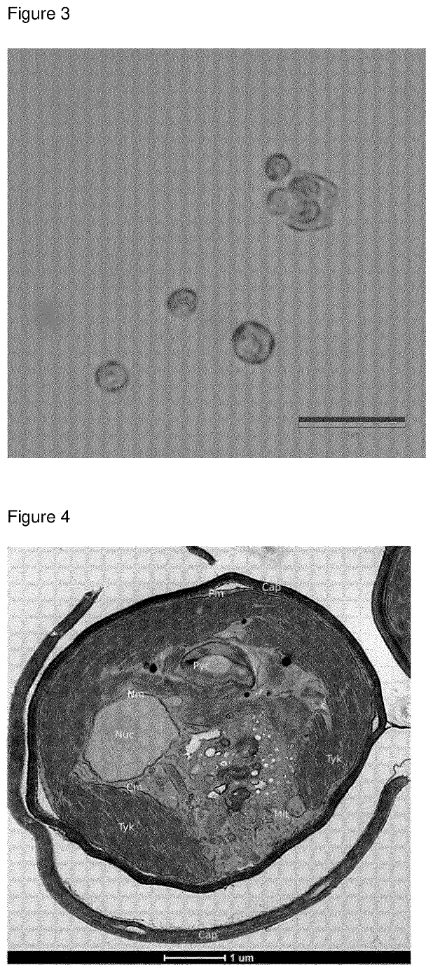

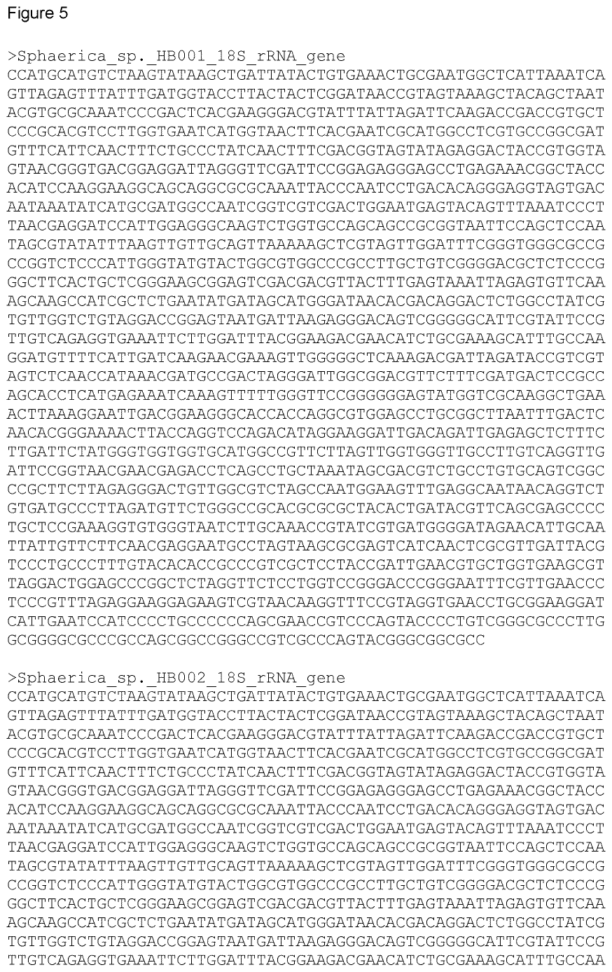

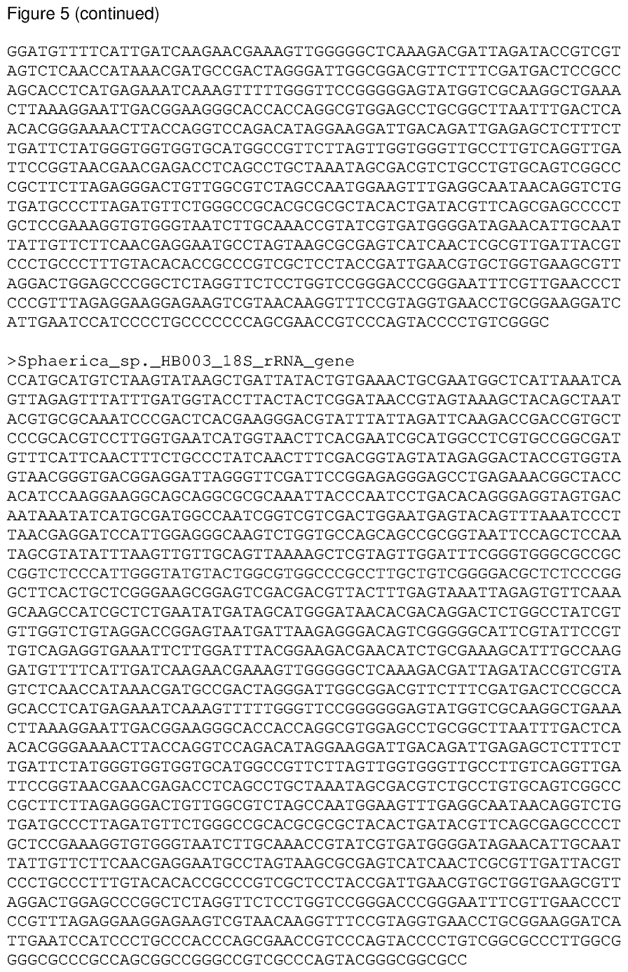

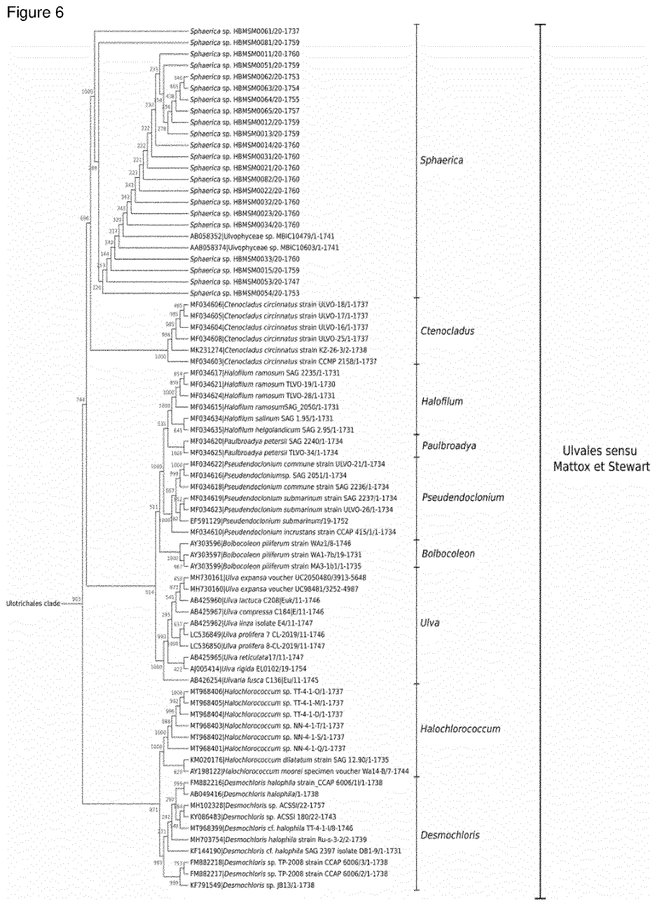

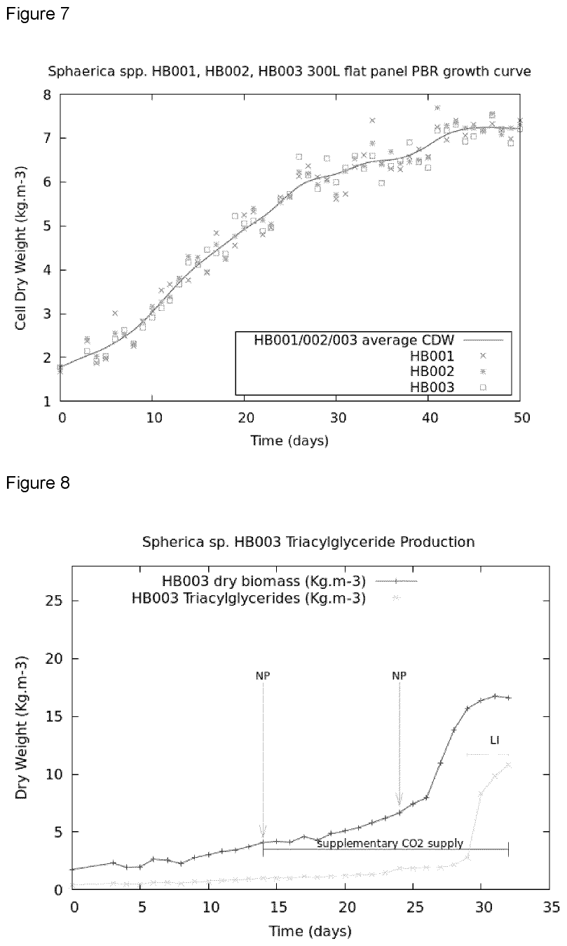

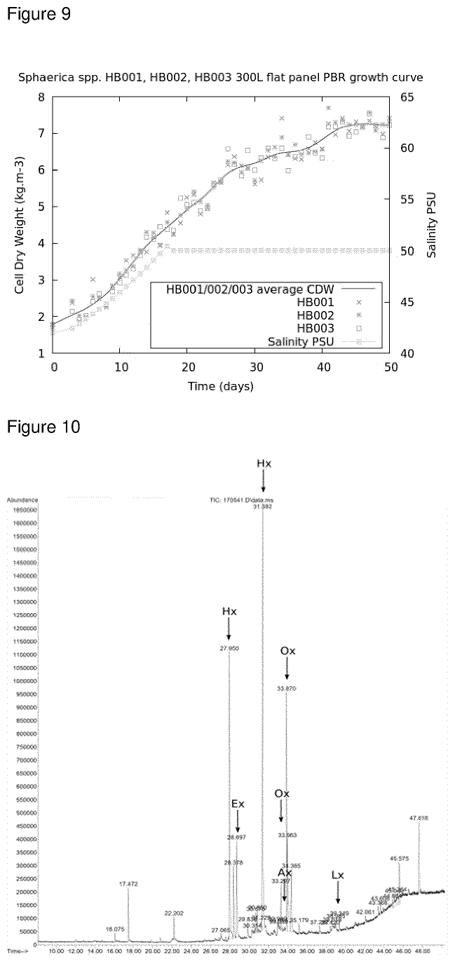

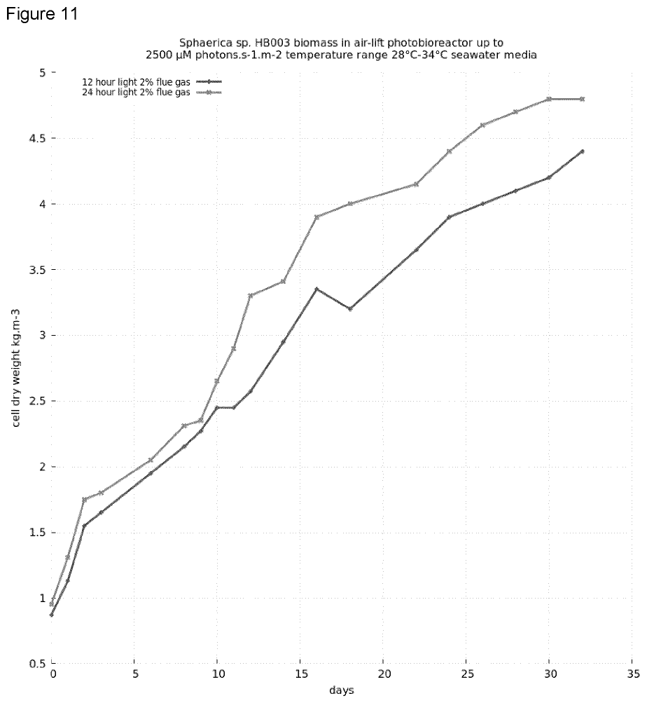

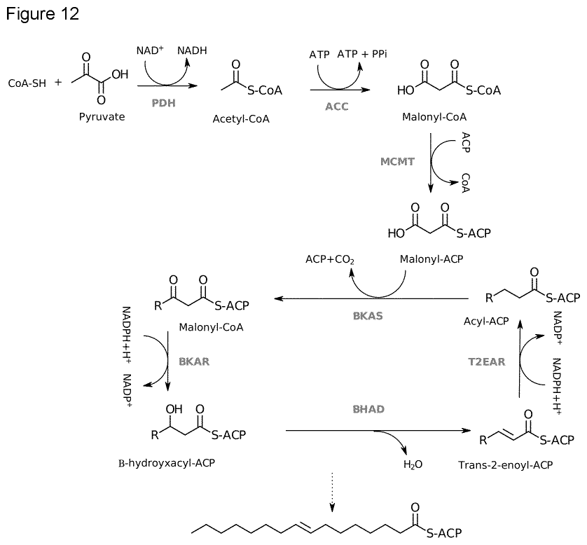

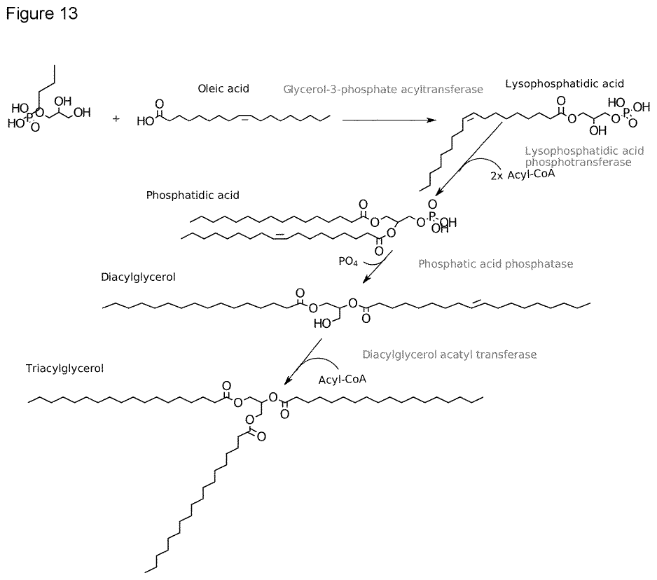

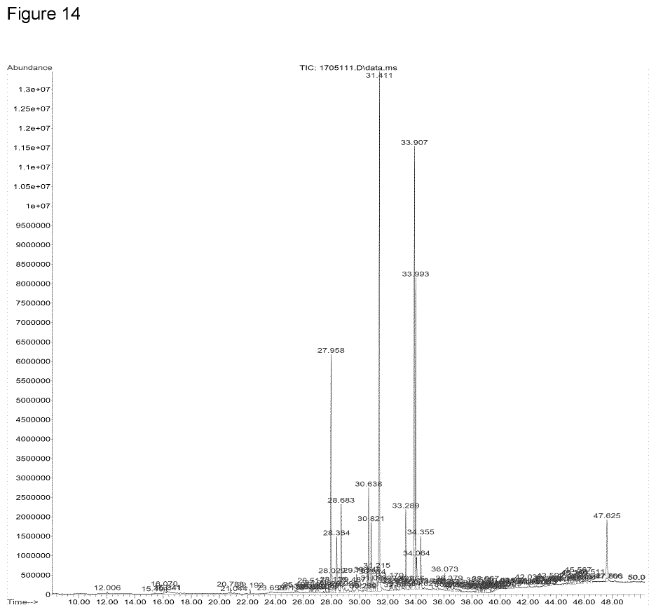

FIG. 3: Bright field image of maternal cell rupturing to release 4 encapsulated non-motile encapsulated daughter cells. Scale bar 10 μM.

FIG. 4 Sphaerica transmission electron microscopy cell ultrastructure.

Sphaerica cell ultrastructure transmission electron micrograph of Sphaerica cell showing; Cap cell capsule; Pm plasma membrane; Nm nuclear membrane; Nuc nucleus; Cm chloroplast membrane; Tyk thylakoids; Mit mitochondria. Cells were fixed with 2.5% glutaraldehyde for 48 h. Osmication was performed using reduced osmium (1:1 mixture of 2% osmium tetroxide and 3% potassium ferrocyanide). After pre-embedding in agar 1%, samples were dehydrated in ethanol series and embedded in epoxy resin. Thin sections (100 nm to 120 nm thickness) were collected on copper grids and contrasted with lead citrate. Imaging was performed using a transmission electron microscope (TEM) operating at 300 kV (Titan Cryo Twin, FEI Company, Hillsboro, OR). Images were recorded on a 4 k×4 k CCD camera (Gatan Inc., Pleasanton, CA).

FIG. 5 Sphaerica 18S rRNA gene sequences for HB001, HB002 and HB003.

FIG. 6 Phylogenetic analysis of Sphaerica HB001, HB002 and HB003.

Neighbour-joining phylogenetic tree, bootstrap 1000 replicates of Sphaerica 18S rRNA small subunit ribosomal RNA. Full length 18S rRNA sequences were progressively aligned within a matrix using ClustalW [97,98]. The guide tree was generated using neighbour-joining algorithm and bootstrapped 1000 times. In terms of their relationship to other taxonomically established algal genera, the monophyletic 18S rRNA sequence analysis places Sphaerica in the Ulvales order of the Ulvophyceae class. The latter encompasses predominantly marine macroalgae with a minority of marine microalgae described. The closest related clade to Sphaerica is Ctenocladus which describes a terrestrial filamentous extremophile microalga.

FIG. 7 Sphaerica HB001, HB002, HB003 cultured in industrial 300 L flat panel Sphaerica HB001, HB002, HB003 cultured in industrial 300 L flat panel PBR; culture medium filtered non-sterile seawater PSU 40 initial-50 PSU final supplemented with FSM medium (without vitamin supplements nor antibiotics nor antifungals; ambient diurnal temperature range 24° C.-35° C. (photobioreactors are not cooled), maximum insolation up to 2100 μM photons·s⁻¹·m⁻² sunlight 12 hours day/12 hour night. Culture purity was assessed microscopically every 48 hours.

FIG. 8 Sphaerica HB003 biomass and lipid production.

Sphaerica HB003 cultured in industrial 300 L flat panel PBR; culture medium filtered seawater supplemented with FSM·diurnal temperature range 24° C.-35° C. maximum insolation 2100 μM photons·s⁻¹·m⁻² day 14-24 2.0% CO₂ day 24-32 5.0% CO₂. Culture purity was assessed microscopically every 48 hours.

FIG. 9 Sphaerica HB003 biomass production under high variable salinity levels.

Sphaerica HB001, HB002, HB003 cultured in industrial 300 L flat panel PBR; culture medium filtered seawater adjusted to 40 PSU initial-50 PSU final supplemented with FSM. Pure CO₂ 2% v/v/m was supplied during daylight. Diurnal temperature range 24° C.-35° C. maximum insolation 2100 μM photons·s⁻¹·m⁻². Salinity rise due to evaporation in the culture medium was measured spectrophotometrically and maintained at or below 50 PSU by addition of filtered seawater.

FIG. 10 Sphaerica total neutral lipid GC-MS analysis.

Gas chromatography-mass spectrometric analysis of derivatised Sphaerica HB003 total neutral lipid extract showing high levels of biofuel triacylglyceride components Hexadecanoic acid trimethylsilyl ester (Hx), Octadecadienoic acid trimethylsilylester (Ox) and; high value polyunsaturated saturated fatty acid derivatives Eicosadienoic acid trimethysilyllester (Ex), Arachidonic acid trimethylsilyl ester (Ax), Linolenic acid trimethysilyllester (Lx).

Lipid samples were incubated at 70° C. for 90 minutes in methoxylamine hydrochloride/pyridine) followed by reaction with N-Methyl-N-(trimethylsilyl)trifluoroacetamide (50 μL) at 70° C. for 60 minutes. Derivatised lipid extracts were analysed on a Perkin Elmer 600 Clarus Gas Chromatogram equipped with Perkin Elmer 5 MS capillary column (30.0 m×0.25 mm ID, 0.25 μm film thickness). The GC conditions were: injector temperature 200° C.; helium as carrier gas with flow rate of 1 ml/minute and a split ratio of 50; and temperature programming. Column temperature was held initially at 40° C. for 1 minutes, and ramped up 10° C. per minute to 50° C. before final temperature increased of 60° C./min to reach 300° C. GC effluent was introduced to the Perkin Elmer 600 Clarus Mass Spectrometer for EI mass spectrometry analysis where data were recorded in full scan or linear mode in the mass range of 50-600 m/z.

FIG. 11 Sphaerica HB003 biomass production on 2% industrial flue gas 12 hour and 24 illumination.

Sphaerica HB003 cultured in 2×450 L large-format flat panel photobioreactor on filtered seawater 38-45 PSU supplemented with FSM. No vitamin nor antibacterials, antifungals. Diurnal temperature range 22° C.-35° C. (no cooling). Maximum insolation 2100 μM photons·s⁻¹·m⁻². During night, for 24 hour culture, 300 μM photons·s⁻¹·m⁻² was supplied from white LED lamps. Industrial flue gas was directly captured and compressed from an incinerator chimney burning coconut shells and husks and palm oil empty fruit bunches. Compressed unfiltered flue gas (~15% CO2) was supplied as 2% v/v/minute during day time 12 hour light condition and continuously during 24 hour light condition. Culture purity was assessed microscopically every 48 hours.

FIG. 12: Sphaerica triacylglyceride synthesis-chloroplast pathway.

FIG. 13: Sphaerica triacylglyceride synthesis-endoplasmic reticulum pathway.

FIG. 14: Example GCMS. The identities of the peaks are set out hereinafter in Table 3 as described in Example 9 and Table 2.

Briefly, analysis of Sphaerica triacylglyceride fatty acid profiles was carried our using cultured Sphaerica HB003 cells grown under nitrogen limitation for 0 hours, 12 hours, 24 hours and 72 hours (condition M3-N1, M3-N2, M3-N3, M3-N4 respectively). The sample condition for FIG. 14 was “M3-N4”

### EXAMPLES

### Example 1-Background to Development of Novel Marine Microalgal Biofuel Production Technology

### Abiotic and Biotic Stressors in Algal Biofuel Production

Microalgal cultures are susceptible to a series of stressors that operate alone or synergistically to impair cellular function. Abiotic light, heat and salinity stress degrade the structure and function of thylakoid membranes and light harvesting systems by disabling proteins, photopigments, lipid membranes and RuBisCo. The consistent effect of these lesions is a reduction in photosynthetic activity, a switch of remaining photosynthetic electron flow from carbon fixation to triacylglyceride metabolism. Biotic stressors such as competitor photoautotrophs, viruses, fungi and zooplankton predators cause rapid loss of productive microalgae and population crash.

Large scale biofuel production imposes multiple biotic and abiotic stresses on the production strain which experience acute changes in light, temperature and salinity that reduce metabolic efficiency below theoretical maximum [10] leading to loss of production [11, 12, 13]. In addition, contamination by other microalgae [14, 15], viral infections [16] fungal parasites [17,18] and predatory zooplankton cause complete crashes of large scale cultures. The effect of these stressors impinge synergistically on biofuel strain productivity.

### Light Stress Responses

Light stress is the central abiotic stressor affecting biofuel yields [11,12,13]. In industrial cultivation systems in which cultures with high optical densities are generated, individual cells are subjected to non-homogenous light distributions as they circulate within the photobioreactor or pond [11,20]. This changeable environment places additional stress on the production cells. In nature, microalgae constantly adjust their light harvesting system to optimise energy harvested under limiting photon fluxes while minimising photodamage at excess photon fluxes. Photosystem II (PSII), photosystem I (PSI) and the cytochrome b6f complexes that are embedded in the thylakoid membrane catalyse the electron flow from water to NADPH coupling proton transfer from stromal to luminal thylakoid membrane face that powers ATP production [21]. In PSII and PSI, light harvesting complexes LHCII and LHCI respectively, harvest photons and transfer the excitation energy to the D1 D2 heterodimer and redox cofactors [22, 23, 24, 25]. Photoinhibition occurs when a strain-specific irradiation level is exceeded. This leads to enhanced levels of photosynthetically generated NADPH and ATP that rapidly exceeds the capacity of the Calvin-Benson reaction leading to saturation of the electron transport chain. This increases the probability of triplet Chlorophylls transferring energy to molecular oxygen forming toxic reactive oxygen species (ROS) [26]. Simultaneously, it raises the trans-thylakoid proton gradient which activates transmembrane proteins LHCSR2 engaging PSII and LHCSR3 engaging PSI switching conformations of both photosystems to a quenching state initiating nonphotochemical quenching (NPQ) dissipating excitation energy as heat [11,27,28]. Luminal acidification induces violaxanthin de-epoxidase (VDE) which converts violaxanthin photopigment into zeaxanthin which is involved in Chl quenching and ROS scavenging [27].

### Light Stress Mitigation

To avoid photoinhibition microalgae adopt three strategies: (1) Stress-related light harvesting complex II subunits LHCSR that are expressed under high light conditions which direct absorbed light to non-photochemical processes reducing the formation of ROS [28,29,30]; (2) reduction in PSII antenna size by phosphorylation-induced dissociation of LHCII protein from PSII, followed by a drop in Chlorophyll b content and reduced expression of LHCII gene expression. These structural and genetic changes enhance photosynthetic efficiency at high light levels because the generation of reactive oxygen species is reduced allowing high steady-state photosynthetic action. ROS damage is thus reduced sufficiently to permit PSII repair cycle to continuously replace damaged reaction centre components [31, 32, 33, 34]; (3) Increased absorption of excitation energy by the synthesis of carotenoids closely associated with PSII and LHCII Chlorophylls. The carotenoids lutein and b-carotene quench excitation state of Chlorophylls preventing reactive oxygen species generation [35,36]. In addition, in the xanthophyll cycle, violaxanthin is converted first to antheraxanthin and then to zeaxanthin which accumulates in PSII and quenches photoexcited Chlorophyll radicals converting the energy to heat [37].

### Thermal Stress

Industrial microalgal cultures outdoors are subject to diurnal and seasonal changes in temperature. While pond cultures can moderate temperature increases through evaporative cooling, at the expense of increases in salinity of up to 20% a day, low volume, high surface area flat panel or tubular photobioreactor systems without temperature management can develop internal temperatures up to 10° C. above ambient in mid afternoon [38]. Characterised biofuel strains isolated from high latitudes exhibit optimal growth under temperate conditions between 20° C. and 24° C. [39]. Under thermal stress, a heat stress response is triggered [40]. Microalgae exhibit species-specific heat stress induction temperatures defined by their ecological niche [41]. Heat stress affects cell function at multiple levels including cell cycle, protein folding/structure/assembly; cell cycle; membrane fluidity; metabolic coordination including triacylglyceride synthesis [42,44]. Heat shock rapidly induces cell cycle arrest in G1 and G2 phases as well as arrest of DNA replication and cell division [45]. Accumulation of misfolded protein induces Hsp nuclear and plastid chaperones [46] and lipid metabolic enzymes phospholipase C and D, phosphatidylinositolphosphate kinase, phosphatidylinositol 4,5-bisphosphate which rapidly increases cellular pool of phosphatidic acid, phosphatidylinositol 4,5-bisphosphate which is converted to diacyl glycerol and inositol 1,4,5-triphosphate [45]. Modulations of membrane fluidity are managed by de novo synthesis of saturated fatty acids [47,48], utilising the increased pool of precursors generated by lipid metabolic enzymes that replace unsaturated fatty acids increasing membrane rigidity [49]. Simultaneously triacylglycerides enriched in polyunsaturated accumulate in lipid bodies [45]. Heat inactivation of Rubisco activase which catalyses the release of ribulose-1,5-bisphosphate from the Rubisco active site reduces CO2 fixation [49] which facilitates a metabolic switch of light reaction NADPH and ATP to fatty acid synthesis and accumulation of lipid droplets [50, 51, 52].

### Salinity Stress Response

Salinity stress retards cell division, reduces cell size, ceases cell motility and induces in some strains palmelloid formation [53,54]. Under salt stress, there is a rapid but reversible osmotic effect as water leaves the cell. In this phase, recovery of PSII and PSI function is dependent on a Na⁺/H⁺ antiporter system that is regulated by membrane fluidity [55,56,57]. If the salt stress extends over hours, irreversible damage to PSII and PSI as well as the Na⁺/H⁺ antiporter system caused by influx of Na⁺ through K⁺/Na⁺ channels. Increasing unsaturation of membrane fatty acids protects PSII and PSI from both rapid reversible and slower irreversible inactivation [57]. At a metabolic level, compatible solutes with neutral charge and low toxicity at high concentration such as glycerol [58-61] trehalose and proline [63] are produced to balance osmotic pressure.

### Biotic Stressors

Large scale microalgal biofuel monoculture is subject to four classes of biological stressors; (1) contamination by other microalgae and cyanobacteria that compete for nutrients and reduce biomass and triacylglyceride productivity [64); (2) viral infections that rapidly crash the microalgal population [65]; (3) fungal parasites such as chytrids [66,67]; (4) zooplankton grazing by ciliates, amoeba, rotifers [68]. Nucleoplasmic large DNA viruses such as prasinoviruses are present at 10⁴·ml⁻¹ in the marine photosynthetic zone and are a significant challenge to large scale microalgal culture [69] causing cell lysis within hours causing a population crash and loss of production [70,71,72]. Acquired virus resistance has been observed among several different microalgal taxa [73, 74, 75, 76], including multi-year resistance [75]. Viral resistance is genetically inherited and a property of particular microalgal strains. Genomic studies of long-term virus resistant Ostreococcus have correlated viral resistance with specific small hypervariable chromosome regions [73, 77, 78].

### Bioprospecting for New Marine Microalgal Biofuel Production Strains

Microalgal strains utilise a set of common stress pathways to manage abiotic and biotic stressors. The trigger points for response are, however, strain-specific as microalgae evolve to optimise cell growth and metabolism over the environmental range of their ecological niche [32]. Therefore, screening algal biodiversity from extreme habitats can identify new strains exhibiting biofuel process-friendly characteristics such as high-light tolerance [79,80]. To date, however, there has been no bioprospecting survey of extreme Tropical marine environments which receive the maximum solar radiation on the planet screening for marine microalgal biofuel strains capable of operating more productively utilising more solar energy than any currently available strains.

### Example 2—Development of Novel Marine Microalgal Biofuel Production Technology

To find new candidate high productivity industrial marine microalgal strains that exhibited process-friendly phenotypes and significantly, synthesised high levels of short chain triacylglycerides that would be suitable for transportation applications without chemical modification, we carried out an extensive, long term bioprospecting program in the South China Sea and Malacca Straits which sampled the huge untapped biodiversity of these seas throughout their seasonal changes [81]. These high-insolation, high-temperature, variable salinity, oligotrophic tropical seas provide an intensive selective environment for highly robust microalgal strains that utilise high level insolation to capture and convert CO₂ into high-energy density bio-oils. Our high throughput bioprospecting survey utilised industrial pumps connected to industrial filtration systems to generate size-fractionated biofilm samples. These biofilms were coupled with multiple abiotic and biotic screening pipelines to select for new high efficiency microalgal candidates capable of growing to high biomass with high drop-in fuel capable triacylglyceride content on seawater, without expensive supplements, antibiotics, antifungals required for previous biofuel strains and resistant to the multiple biotic and abiotic stressors.

### Bioprospecting Pipeline

Our bioprospecting programme screened marine microalgal isolates from the South China Sea and the Malacca Straits in a massively parallel abiotic and biotic multiple stress selection and isolation pipeline to generate an industrial strain library comprising over 100 physiologically robust, high efficiency marine strains that thrive under high light, temperature and salinity conditions. Microalgal samples from marine surface layer (2 m) were obtained from multiple locations throughout the year in each sea in a long-term bioprospecting programme. Microalgae were extracted by high volume sequential filtration at 15 μM, 5 μM, 1 μM and 0.1 μM. Biofilm cultures obtained were subjected to multiple stressor selection by incubation in filtered seawater supplemented with vitamin-free nitrogen sources, phosphorous and trace elements at 30° C.-35° C., 100-500 μM photons·s⁻¹·m⁻², 35-40 PSU. During this primary growth phase, biotic stressors particularly viruses and predatory bacteria and zooplankton act as selective agents for highly resistant microalgae [82, 83, 84, 85, 86, 87]. Pure microalgal cultures were generated by micromanipulation and dilution culture.

### Targeted Phenotypes

Our bioprospecting pipeline targeted five key phenotypic characteristics which we consider to be required for an economically viable, robust and scalable industrial biofuel microalgal production system that maximises triacylglyceride production. These were: (1) Multiple independent fast-growing, high biomass-accumulating isolates that rapidly and routinely generate high biomass (>5 g·l⁻³ cell dry weight) with a high triacylglyceride content (>40% cell dry weight) of a biofuel compatible chain length profile (<C22) on simple seawater based media without vitamin supplements allowing rapid, high and cost-effective triacylglyceride harvest cycles; (2) Multiple independent high light-tolerant strains that can efficiently utilize high photon fluxes (>2000 photons·s⁻¹·m⁻²) with minimal photoinhibition; (3) Multiple independent thermo-tolerant strains that grow well between 20° C. and 38° C. and that remain viable at 45° C. enabling efficient operation at Tropical temperature ranges; (4) Multiple independent high salinity tolerant strains capable of rapid and efficient growth and triacylglyceride accumulation across a salinity range of 35 PSU to 40 PSU removing the need for expensive freshwater to control salinity which otherwise rapidly increases as a result of evaporation; (5) Multiple independent isolates to prevent biotic stressors (viruses, grazers and predators) attacking a single microalgal clone and causing population crashes [82, 83, 84, 85, 86, 87].

### Example 3—Isolation and Directed Evolution Engineering of Sphaerica

Our bioprospecting programme generated an industrial strain library comprising over 100 physiologically robust, highly productive marine strains that thrive under high light, temperature and salinity conditions and exhibit strong resistance to biotic stressors. From this library, using quantitative physiological ranking to determine the optimal strains, we identified a group of unicellular marine microalgae present in all three environments that subsequent monophyletic taxonomic analysis were shown to be a completely new genus of tropical marine microalgae that is endemic to all three tropical sea locations. We have named this new Chlorophyte genus Sphaerica.

### Sphaerica Cell Structure

Light microscopic analysis (FIG. 1-3) shows that Sphaerica vegetative cells, are non-motile, have no cilia nor flagellae, are uninucleate, spherical and solitary, with a smooth cell wall and capsule. The chloroplast is parietal with penta-radially symmetrical lobes and a single pyrenoid. The chloroplast is remarkable in that it possesses a very large number of thylakoids. Each chloroplast contains up to ~300 thylakoid membranes (range 120-298 thylakoid membranes/cell, n=1430) running parallel to cell wall. The single nucleus is completely surrounded by chloroplast which can afford significant protection to UV radiation. The cell can contain over 60% cell dry mass as lipid. Multiple lipid bodies are displaced to lower hemisphere. Asexual reproduction occurs in two rounds of division (FIG. 1-3); the second division plane is at 90° to first division. All divisions are contained in the maternal cell capsule. Autospores (4) are non-motile, each with a cell wall and capsule. This gives a growth rate of ((4n)×0.25) i.e. 4, 16, 64, 256. Sexual reproduction has not been observed.

The Sphaerica cell (FIG. 1-3) range in size from ~1.52 μm to 10.28 μm (n=427); nascent daughter cells range from 1.52 μm to 2.50 μm, mature cells range from 2.50 μm to 6.00 μm and maternal cells range from 6.00 μm to 10.28 μm in diameter.

The cells are always spherical or near spherical in structure being enclosed within a polysaccharide capsule that ranges from 0.215 μm to 0.344 μm in thickness (with an average of 0.277 μm (n=35)). This provides a permeable, predator-resistant layer around the cell membrane. The cell itself is dominated by the chloroplast which occupies approximately 40% of the cell volume. It is a cup-shaped often penta-lobed, parietal structure that surrounds nucleus, pyrenoid and mitochondria. The Sphaerica chloroplast is distinguished by the huge number of thylakoid layers within the chloroplast. Each Sphaerica contains up to ~300 thylakoid membranes. The thylakoids enclose an approximately 17 nm wide lumen and form a continuous structure throughout the chloroplast with thylakoids running parallel to the cell membrane. The very large number of thylakoid membranes s facilitates Sphaerica's high light tolerance as overlapping thylakoids can attenuate light penetration under high light conditions minimising photoinhibition of thylakoids deeper within the chloroplast. Intimately linked to the thylakoid layer is the 1.17 μm×0.64 μm ovoid pyrenoid. This is a non-membrane-bound phase-separated subcellular organelle associated with the chloroplast stroma [88] which contains carbonic anhydrase [89] RuBisCO activase [90] and tightly organised arrays of RubisCO [88] which together act as a cellular CO₂ concentrating and fixation system [91,92,93]. The pyrenoid communicates with thylakoid lumen via microtubules [88]. The chloroplast also encloses the 1 μm×0.8 μm ovoid, membrane bound nucleus and the lipid vesicles, which are membrane bound triacylglyceride storage organelles, are located beneath and between the chloroplast lobes, in the lower hemisphere of the cell.

### Sphaerica Genus Description

Sphaerica genus cells are unicellular, spherical, non-motile, ranging from ~1.52 μm to ~10.28 μm. The chloroplast is bright green and parietal that occupies one hemisphere of the cell with lobes extending into the remaining hemisphere which contains lipid vacuoles. Cells are surrounded by a 0.215 μm to 0.344 μm thick polysaccharide capsule which expands as the cell grows and within which cell division occurs. Ultrastructure studies show mature Sphaerica cells are enclosed by a spherical polysaccharide capsule that provides a permeable, predator-resistant outer cell envelope. Approximately 40% of cellular volume is occupied by the often penta-lobed parietal chloroplast that enfolds nucleus, pyrenoid and mitochondria subcellular compartments. The chloroplast is distinguished by the very high number of thylakoids membranes (~300 per cell).

### Sphaerica Taxonomic Analysis

The taxonomic position of selected Sphaerica isolates we obtained was established as a new and distinct genus within the Ulvophycean order Ulvales based on phylogenetic analysis of full length 18S rRNA genes (1770 bp) (FIG. 5). The Ulvophyceae are one of the four classes of the Chlorophyta and it encompasses the greatest morphological and cytological diversity within the Kingdom. Ulvophyceae is comprised of eight orders that have been classically defined on life cycle and ultrastructural elements, and presently this has been further defined by molecular phylogenetic approaches. The order Ulvales, within which Sphaerica form a new genus, is a distinct clade supported by nuclear and plastid gene phylogenies [94,95]. The Ulvales range from unicellular to filamentous microalgal forms to macroalgal seaweeds.

The Ulvales order itself is a polyphyletic clade with clear evidence of horizontal gene transfer of rRNA loci between macro and micro algal members. Although primarily marine, some freshwater species are described. Molecular phylogenies indicate that macroalgal Ulvophyceae evolved in multiple lineages independently from simple unicellular or filamentous forms [96]. Our isolation physiological and genomic characterisation of Sphaerica provides a benchmark in our understanding of this poorly characterised group.

18S rRNA monophyletic analysis of Sphaerica HB001, HB002 and HB003 show that their closest phylogenetic genus is the extremophile terrestrial filamentous microalga Ctenocladus genus. This is a rarely found filamentous microalgae that replicates through filament breakage into akinetes [Blinn D. W., Stein, J. R., 1970. Distribution and taxonomic reappraisal of Ctenocladus circinnatus (Chlorophyceae, Chartophorales) J. Phycol. 6 101-105.]. The physical appearance, cell form and life cycle of Sphaerica and Ctenocladus are radically different which is consistent independent genus status for both clades. More distant monophyletic relationships are observed with five other Ulvales genera; Halophilum, Paulbroadya, Pseudoendococlonium, Bolbocoleon and Ulva. Once again, on the basis of cell form, and, life cycle, Sphaerica is clearly a distinct genus from these organisms: The extremophile Halophilum is a photobiont of the lichen forming fungas Verrucaria serpuloides. Halophilum is a short-branched disintegrating filamentous high latitude marine microalgae [Darienko, T., Roschuld, T. 2017. Toward a monograph of non-marine Ulvophyceae using an interative approach Phytotaxa 324 (1), 001-041.]. Paulbroadya, a richly filamentous marine microalgae found attached to Antarctic rock surfaces with up to four thylakoid membranes within its single parietal chloroplast [Paul A. Broady & Manfred Ingerfeld (1993) Three new species and a new record of chaetophoracean (Chlorophyta) algae from terrestrial habitats in Antarctica, EuropeanJournal of Phycology, 28:1, 25-31, DOI: 10.1080/09670269300650041]. Pseudoendoclonium is a poorly described high latitude terrestrial filamentous microalgae found on barnacle and rock surfaces [T. Edelstein and J. Mclachlan 1967 Investigations of the marine algae of nova scotia: iv. species of chlorophyceae new or rare to Nova Scotia Candadian Journal of Botany 45 (2); Tupa, D. D. 1974. An investigation of certain chaetophoralean algae. Beih. zur Nova Hedwigia 46 (Suppl.): 64-67.] and as an epiphyte [https://ethos.bl.uk/OrderDetails.do?uin=uk.bl.ethos.297830]. Bolbocoleon are marine filamentous microalgae that are epiphytic on marine seagrasses [Karl Gunnarsson and Ruth Nielsen 2016.Culture and field studies of Ulvellaceae and other microfilamentous green seaweeds in subarctic and arctic waters around Iceland 2016 Nova Hedwigia 103 (1): 17-46 DOI: 10.1127/nova_hedwigia/2016/0334S]. Ulva are benthic marine macroalgae up to 1 m long present in high latitude coastal and inland saline lakes [. Hayden, H. S., J. Blomster, C. A. Maggs, P. C. Silva, M. J. Stanhope, and J. R. Waaland. 2003. Linnaeus was right all along: Ulva and Enteromorpha are not distinct genera. European Journal of Phycology 38:277-294].

### Consideration of AB058352 and AB058374

DNA-DNA sequence homology searches of ncbi non-redundant sequence database with HB001 full length 18S rRNA locus identified one sequence entry “AB058352.1:1-1746 Ulvophyceae sp. MBIC10479 gene for 18S rRNA, partial sequence” dating from 2001.

This 1746 nt 18S rRNA partial sequence fragment submission that exhibited 100% DNA:DNA homology with HB001 18S rRNA locus 1-1746 bp 18S rRNA subregion.

Further investigation of the ncbi database revealed that a second sequence entry, with an identical sequence. This sequence entry “AB058374” was an also defined as an unidentified member of the Ulvophyceae class.

The source organisms for these partial sequences were seemingly not deposited and have proved to be unavailable from any other source, as is further information concerning their full sequences, or any phenotypic information (for example structural, ultrastructural and metabolic etc). Therefore the limited information given in the sequence databases unfortunately cannot be confirmed or supplemented in any way.

### Example 4—Generating Optimised Biofuel Production Strains

Biofuel production typically demands large scale water resources [100, 101, 102, 103], including blowdown freshwater addition for marine microalgal culture [104, 105]. Under outdoor Tropical conditions, typical maximum of ~2100 μM photons·s⁻¹·m⁻², 20° C.-35° C. ambient temperatures, we recorded 2.4% daily increase in salinity in our flat panel photobioreactor systems. Over a 30-day period, culture media salinity rose from 32 PSU to 60 PSU. In open ponds, under the same conditions, salinity rose 20% per day. This salinity rise is usually controlled by adding blowdown freshwater incurring large operational costs and unsustainable use of precious freshwater resources [101, 104, 105, 106]. To develop Sphaerica biofuel production strains that minimise operational costs and that do not require blowdown freshwater addition, multiple genetic loci must be altered [60,61,63]. Therefore, we applied directed evolution techniques to generate high salinity tolerant high biomass and triacylglyceride Sphaerica production strains [107]. Random UV mutagenesis of wild type Sphaerica strains was carried out in which UV dosage was empirically determined (viability was reduced to ~50%) and the mutagenised population was challenged with augmented salinity media to select for derivatives with increased tolerance to high-salinity media. In a second round of mutagenesis and selection, high flux carbon uptake Sphaerica strains were constructed from high-salinity strains by directed evolution enabling them to efficiently grow in 15% CO₂ (either CO2 gas or agricultural or industrial flue gas) which figure is directly compatible with industrial flue gas CO₂ content. Using this approach, we successfully generated a library of optimised Sphaerica production strains including preferred Sphaerica biofuel production strains HB001, HB002 and HB003.

Optimised lead Sphaerica biofuel production strain physiological performance envelope (for Sphaerica HB001, HB002 and HB003) is summarised in Table 1. These optimised production strains exhibit exceptionally robust physiologies as a result of multiple genetic changes.

TABLE 1
Sphaerica optimised biofuel strain performance summary

| Character | Data |
|---|---|
| Biomass max 6.5% PE | 16 kg · m − 3 cell dry weight |
| Triacylglycerides max | 10.4 kg · m − 3 |
| Polyunsaturated fatty acids | Eicosadienoic acid, Arachidonic acid, |
| | Linolenic acid and others |
| Photon flux max | up to 2100 μM photons · s − 1 · m − 2 |
| Active temperature range | 4° C.-45° C. |
| Salinity tolerance range | PSU 15-60 |
| Growth medium | Filtered non sterile seawater |
| Vitamin growth supplements | None |
| Freshwater requirements | None |
| Carbon sources | Industrial flue gas (15% CO₂); biogas CO₂ |
| Nitrogen sources | NO3, CH4N2O |
| Phosphate sources | PO4 |
| pH operational range | pH 6.5-pH 11.0 optimum pH 8.2 |
| Genome sequence, gene | 31.8 Mb 25,000 gene annotations |
| transcriptional and | |
| functional map | |

### Example 5—Product Classes from Sphaerica

### Triacylglyceride Bio-Oil

The Sphaerica bio-oil product comprising TAG and PUFA can be directly employed in a diesel fuel without hydrotreatment or transesterification. This high energy density liquid fuel provides superior energy density characteristics than bioethanol. Sustainable aviation fuel can also be generated by simple chemical modification.

### Polyunsaturated Fatty Acids

Sphaerica produce high value polyunsaturated saturated fatty acids (observed as derivatives Eicosadienoic acid, Arachidonic acid, and Linolenic acid.

### High Protein Feed

Sphaerica biomass provides a high quality high protein agricultural feedstock.

### Example 6—Triacylglyceride Synthesis in Sphaerica

Triacylglyceride synthesis in microalgae incorporates malonyl molecules in two cyclic, multi-enzyme catalysed stages initiating in the chloroplast and following translocation out of the chloroplast, completing in the endoplasmic reticulum (FIGS. 12 and 13).

Sphaerica triacylglyceride biosynthesis initiates with fatty acid synthesis in the microalgal chloroplast stroma (FIG. 12). The first reaction is the formation of acetyl-CoA catalysed by pyruvate dehydrogenase (PDH) which plays a key role in carbon flux through the pathway. Chloroplast pyruvate dehydrogenase oxidatively decarboxylates pyruvate to generate acetyl-CoA and NADH [1].

Expression of PDH is upregulated under nitrogen starvation which induces TAG accumulation [2,3]; Gene silencing of chloroplast E1a PDH subunit reduced total fatty acid production by 40% [4]. Acetyl-CoA synthetase (ACC) catalyses the first committed reaction in TAG synthesis pathway forming Malonyl-CoA [5] chloroplast ACC in Chlorophyta is a heteromeric multisubunit complex of biotin carboxylase (BC), biotin carboxyl carrier protein (BCCP) and a and b-carboxyltransferase (a-b-CT) [6] that catalyses the formation of malonyl-CoA in a two-step reaction powered by ATP. MCMT Malonyl-CoA:ACP malonyltransferase converts malonyl-Co to malonyl-acyl carrier protein. Overexpression of MCMT gene increases neutral lipid content by 30% and improves growth rate and photosynthetic efficiency [7]. Fatty acid chain elongation occurs by sequential addition of malonyl-ACP by ligation to acetyl-CoA forming 3-ketoacyl-ACP by ketoacyl-ACP synthase (KAS) with the release of CO2. The 4-carbon 3-ketoacyl-ACP is reduced by ketoacyl-ACP reductase (KAR), dehydrated by hydroxyacyl-ACP dehdrase (HD), reduced again by enoyl-ACP reductase (ER) generating a 6 carbon ACP chain product. KAS-KAR-HD-ER form a multi-subunit bacterial type II fatty acid synthase (FAS) complex [8]. Seven FAS reaction cycles generates C16 fatty acid chain length products which is released by acyl-ACP thioesterase (FAT). Overexpression of FAT increases total fatty acid content [9,10]. Nascent fatty acids are transported from the chloroplast in an as yet unknown transport pathway to the cytoplasm where triacylglycerides are generated in the Kennedy pathway. In these reactions, glycerol-3-phosphate is acylated by glycerol 3-phosphate acyltransferase (GPAT) forming lyso-phosphatididc acid, which is converted to phosphatidic acid by lysophosphatidic acid acyltransferase (LPAT). Phosphatidic acid is dephosphorylated to form diacylglycerol. The final step in the TAG pathway is catalysed by diacylglycerol acytransferase (DGAT) which generates the TAG carbon-energy storage molecule [11, 12].

### Example 6 References

(1) Mooney, B. P.; Miernyk, J. A.; Randall, D. D. Cloning and Characterization of the Dihydrolipoamide S-Acetyltransferase Subunit of the Plastid Pyruvate Dehydrogenase Complex (E2) from Arabidopsis. Plant Physiol. 1999, 120 (2), 443. https://doi.org/10.1104/PP.120.2.443.
(2) O, A.; A, B.; I, R.; U, P. Enhanced Acetyl-CoA Production Is Associated with Increased Triglyceride Accumulation in the Green Alga Chlorella Desiccata. J. Exp. Bot. 2015, 66 (13), 3725-3735. https://doi.org/10.1093/JXB/ERV166.
(3) Miller, R.; Wu, G.; Deshpande, R. R.; Vieler, A.; Gärtner, K.; Li, X.; Moellering, E. R.; Zäuner, S.; Cornish, A. J.; Liu, B.; Bullard, B.; Sears, B. B.; Kuo, M.-H.; Hegg, E. L.; Shachar-Hill, Y.; Shiu, S.-H.; Benning, C. Changes in Transcript Abundance in Chlamydomonas Reinhardtii Following Nitrogen Deprivation Predict Diversion of Metabolism. Plant Physiol. 2010, 154 (4), 1737-1752. https://doi.org/10.1104/PP.110.165159.
(4) Shtaida, N.; Khozin-Goldberg, I.; Boussiba, S. The Role of Pyruvate Hub Enzymes in Supplying Carbon Precursors for Fatty Acid Synthesis in Photosynthetic Microalgae. Photosynthesis Research. Kluwer Academic Publishers Sep. 17, 2015, pp 407-422. https://doi.org/10.1007/s11120-015-0136-7.
(5) Li-Beisson, Y.; Thelen, J. J.; Fedosejevs, E.; Harwood, J. L. The Lipid Biochemistry of Eukaryotic Algae. Progress in Lipid Research. Elsevier Ltd Apr. 1, 2019, pp 31-68. https://doi.org/10.1016/j.plipres.2019.01.003.
(6) Hu, Q.; Sommerfeld, M.; Jarvis, E.; Ghirardi, M.; Posewitz, M.; Seibert, M.; Darzins, A. Microalgal Triacylglycerols as Feedstocks for Biofuel Production: Perspectives and Advances. Plant Journal. May 2008, pp 621-639. https://doi.org/10.1111/j. 1365-313X.2008.03492.x.
(7) Chen, J. W.; Liu, W. J.; Hu, D. X.; Wang, X.; Balamurugan, S.; Alimujiang, A.; Yang, W. D.; Liu, J. S.; Li, H. Y. Identification of a Malonyl CoA-Acyl Carrier Protein Transacylase and Its Regulatory Role in Fatty Acid Biosynthesis in Oleaginous Microalga Nannochloropsis Oceanica. Biotechnol. Appl. Biochem. 2017, 64 (5), 620-626. https://doi.org/10.1002/bab.1531.
(8) Li-Beisson, Y.; Shorrosh, B.; Beisson, F.; Andersson, M. X.; Arondel, V.; Bates, P. D.; Baud, S.; Bird, D.; DeBono, A.; Durrett, T. P.; Franke, R. B.; Graham, I. A.; Katayama, K.; Kelly, A. A.; Larson, T.; Markham, J. E.; Miquel, M.; Molina, I.; Nishida, I.; Rowland, O.; Samuels, L.; Schmid, K. M.; Wada, H.; Welti, R.; Xu, C.; Zallot, R.; Ohlrogge, J. Acyl-Lipid Metabolism. Arab. B. 2010, 8, e0133. https://doi.org/10.1199/tab.0133.
(9) Y, G.; X, G.; X, W.; Z, L.; M, J. Characterization of a Novel Thioesterase (PtTE) from Phaeodactylum Tricornutum. J. Basic Microbiol. 2011, 51 (6), 666-672. https://doi.org/10.1002/JOBM.201000520.
(10) Xiahui Hao, Ling Luo, Juliette Jouhet, Fabrice Rébeillé, Eric Maréchal, Hanhua Hu, Yufang Pan, Xiaoming Tan, Zhuo Chen, Lingjie You, Hong Chen, Fang Wei & Yangmin Gong Enhanced Triacylglycerol Production in the Diatom Phaeodactylum Tricornutum by Inactivation of a Hotdog-Fold Thioesterase Gene Using TALEN-Based Targeted Mutagenesis. Biotechnol. Biofuels 2018, 11 (1). https://doi.org/10.1186/S13068-018-1309-3.
(11) Banerjee, S.; Mudliar, S.; Sen, R.; Giri, B.; Satpute, D.; Chakrabarti, T.; Pandey, R. A. Commercializing Lignocellulosic Bioethanol: Technology Bottlenecks and Possible Remedies. Biofuels, Bioproducts and Biorefining. January 2010, pp 77-93. https://doi.org/10.1002/bbb.188.
(12) Ng, I. S.; Tan, S. I.; Kao, P. H.; Chang, Y. K.; Chang, J. S. Recent Developments on Genetic Engineering of Microalgae for Biofuels and Bio-Based Chemicals. Biotechnology Journal. Wiley-VCH Verlag Oct. 1, 2017. https://doi.org/10.1002/biot.201600644.

### Example 7—Sphaerica Industrial Triacylglyceride Production Protocol

### a) Media Compositions

Non-sterile, seawater media supplemented with non-sterile nitrogen, phosphorous and trace element FSM medium prepared as a non-sterile stock as follows:

### FSM100

NaNO₃ 70.5926 mM, NaH₂PO₄·2H₂O 3.2611 mM, C₁₀H₁₄N₂Na₂O₈·2H₂O 0.2594 mM, FeCl₃·6H₂O 0.2331 mM, CuSO₄·5H₂O 0.0012 mM, ZnSO₄·7H₂O 0.0015 mM, CoCl₂·6H₂O 0.0008 mM, MnCl₂·4H₂O 0.0182 mM, Na₂MoO₄·2H₂O 0.0005 mM

### FSM10

NaNO3 7.0592636952657 mM, NaH2PO4·H2O 0.326110587723748 mM, FeCl3·6H2O 0.02330743618202 mM, Na2EDTA 0.0259361708456024 mM, CuSO4·5H2O 7.85004806151874E-05 mM, ZnSO4·7H2O 0.000152990264255911 mM, COCl2·6H2O 0.000154036922650359 mM, MnCl2·4H2O 0.00181891673403395 mM, Na2MoO4·2H2O 5.2073065950538E-05 mM.

### FSM5

NaNO3 3.52963184763285 mM, NaH2PO4·H2O 0.163055293861874 mM, FeCl3·6H2O 0.01165371809101 mM, Na2EDTA 0.0129680854228012 mM, CuSO4.5H2O 3.92502403075937E-05 mM ZnSO4·7H2O 7.6495 1321279555E-05 mM COCl2.6H2O 7.70184613251795E-05 mM, MnCl2·4H2O 0.000909458367016975 mM, Na2MoO4·2H2O 2.6036532975269E-05 mM

### b) Example Sphaerica Production Culture Protocols at Different Scales

### 1 Litre Stage Inoculum

300 ml FSM10 medium inoculated with 50 ml Sphaerica culture generated from pure starter culture in five 2.8 L airlift photobioreactor. culture volume increased to 1 L by addition of FSM10 media 21 days 3 kLux·m⁻² illumination (LED white light) 32° C. Culture growth followed spectrophotometrically and by cell dry weight measurements.

### 10 Litre Stage Inoculum

Ten 5 Litre FSM10 medium inoculated with 1 L Sphaerica inoculum culture to initial concentration of 1 g·L⁻¹ in ten 15 L airlift photobioreactors. Culture volume increased to 10 L by addition of FSM10. Culture incubated 21 days 10 kLux·m⁻² illumination (LED white light) 32° C. Culture growth followed spectrophotometrically and by cell dry weight measurements.

### 300 Litre Stage Inoculum

150 L of FSM5 medium inoculated with 50 L inoculum to initial concentration of 1 g·L⁻¹. Culture volume increased to 300 L by gradual addition of FSM5 medium. Aeration approximately 0.5 vol·vol⁻¹·min⁻¹ with atmospheric air. CO² supplied from industrial flue gas (approximately 15% vol·vol⁻¹ CO₂) supplied at approximately 1.6% vol·vol⁻¹·min⁻¹. Culture incubated 30 days in sunlight up to approximately 105 kLux typically 25° C.-35° C. Culture growth followed spectrophotometrically and by cell dry weight measurements.

### 1000 Litre Production Culture

700 L of FSM5 medium inoculated with 300 L inoculum to initial concentration of 1 g·L⁻¹. Aeration approximately 0.5 vol·vol⁻¹·min⁻¹ with atmospheric air. CO² supplied from industrial flue gas (approximately 15% vol·vol⁻¹·min⁻¹ CO₂) supplied at approximately 1.6% vol·vol⁻¹·min⁻¹. Culture incubated 30 days in sunlight up to approximately 105 kLux typically 25° C.-35° C. Culture growth followed spectrophotometrically and by cell dry weight measurements.

### Example 8—Triacylglyceride Extraction Process

After around 30 days incubation, a low nitrogen high carbon ratio environment induces high level triacylglyceride synthesis. The triacylglyceride “bio-oil” may be extracted according to the following protocol:
Stop the aeration inside 1000 L PBR to let the cell biomass settle.
The settled cell slurry (viscosity 5-10 cP) is pumped from the bottom of the PBR and directed to the filtration unit (inclined nylon mesh 500 (25-28 μm pore size).
Concentrated cell mass (700-800 cP; ~80% biomass is retained by filtration, ~20% biomass mostly nascent cells is returned to the PBR to continue culture). Concentrated cell mass can be stored at 4° C. or directly directed to cell lysis.
Lysis is carried out using high speed industrial homogeniser (5700 rpm 90 minutes 30° C.-32° C.) in the presence of 40% 0.5 and 1.0 mm glass beads in the presence of methanol (equal volume so 50% methanol).
Following lysis glass beads are allowed to sediment and supernatant (lysed cellular material) is transferred to another vessel and mixed with equal volume of hexane. The mixture is mixed using a high speed disperser unit at 3000 rpm 10 minutes 30° C.-32° C.
Phases are resolved by brief centrifugation 4000 rpm 5 minutes room temperature.
Hexane triacylglyceride layer is recovered and triacylglyceride purified by mild heating/distillation to remove hexane leaving the triacylglycerides.
(Methanol and hexane are recycled)

### Example 9—Analysis of Sphaerica Triacylglyceride Metabolic Profile and Accumulation

Detailed metabolic analysis of microalgae has recently benefited from advances in GC-MS database/interpretation techniques (Beale, D. J., Pinu, F. R., Kouremenos, K. A. et al. Review of recent developments in GC-MS approaches to metabolomics-based research. Metabolomics 14, 152 (2018). https://doi.org/10.1007/s11306-018-1449-2).

Standard GC-MS analysis derivatises triacylglycerides to their corresponding fatty acid methyl esters (FAME). Molecular identities are determined by bioinformatic analysis of mass-spectra of molecular fragments. This technique is rapidly evolving, and in particular, the breadth of molecular fragment coverage of the databases is expanding.

The total mass of triacylglycerides synthesised and their molecular identities are a feature of the individual producing microalga which determine the carbon-flux from photosynthesis into triacylglyceride synthesis as well as the molecular profile of triacylglyceride molecules themselves.

### GC-MS Spectra Analysis of Sphaerica Triacylglyceride-Fatty Acid Profiles

The analysis is based on the profile of Sphaerica HB003 cells in an induction (nitrogen limitation) time curve extending from t 0 hours, 12 hours, 24 hours and 72 hours (M3-N1, M3-N2, M3-N3, M3-N4 respectively).

For this analysis FSM 100 medium was used with a working volume of 10 L. FSM100 medium is non-sterile seawater containing: NaNO3 70.592636952657 mM, NaH2PO4·H2O 3.26110587723748 mM, FeCl3·6H2O 0.2330743618202 mM, Na2EDTA 0.259361708456024 mM, CuSO4·5H2O 0.000785004806151874 mM, ZnSO4·7H2O 0.00152990264255911 mM, COCl2·6H2O 0.00154036922650359 mM, MnCl2·4H2O, 0.0181891673403395 mM, Na2MoO4·2H2O 0.00052073065950538 mM

The following protocol was used:
At day 7: increase the working volume from 5 to 10 L.
At day 22: add concentrated FSM 100 media into the 10 L culture
At 4 to 5 day interval: add sterile water to replace the evaporated water.

The growth duration of culture at 10 L working volume is about 30 days.

### GC-MS Sample Preparation

At the end of cultivation period, cells were subjected to stress conditions to induced lipid production. This was achieved as follows:
1 Cells were allowed to settle down by turning off the aeration. Most of the liquid was removed using a peristaltic pump. Cells were washed using sterile sea water which was then also pumped out.
2a For bag A, as a control, 10 L FSM 100 was added.
2b For bag B, as an N-starvation (induction) condition, 10 L FSM 100 media without the nitrogen sources was added.
3 The sampling was done as follow:

| | | Volume | Control | | Nitrogen starvation | |
|---|---|---|---|---|---|---|
| | | of | Weight of | Weight of | Weight of | Weight of |
| Day | Hours | sample | wet cell | dry cell | wet cell | dry cell |
| 0 | 0 | 1 | 13.317 g | 1.3 g | 11.139 g | 1.1 g |
| ½ | 12 | 3 | 40.201 g | 4.0 g | 35.904 g | 3.5 g |
| 1 | 24 | 3 | 48.626 g | 4.8 g | 43.842 g | 4.3 g |
| 3 | 72 | 3 | 48.094 g | 4.8 g | 49.014 g | 4.9 g |

4 Lipid extraction of wet biomass was done by using chloroform:methanol: water (2:2:0.8) method. And vortex with bead beating for 20 to 30 minutes. Centrifuge at 4000 rpm for 3 minutes and collect the chloroform layer.

| Code | Sample |
|---|---|
| M3-C1 | CONTROL - DAY 0 |
| M3-C2 | CONTROL - DAY 1/2 |
| M3-C3 | CONTROL - DAY 1 |
| M3-C4 | CONTROL - DAY 3 |
| M3-N1 | N STARVATION - DAY 0 |
| M3-N2 | N STARVATION - DAY 1/2 |
| M3-N3 | N STARVATION - DAY 1 |
| M3-N4 | N STARVATION - DAY 3 |

5 For further comparison the TAG lipid production by strains HBMSM 001, HBMSM 002 and HBMSM 003 were compared. Strains were cultured in 25 cm diameter polyethylene bags in FSM10/FSM5 under LED and driving mass transfer by air sparging. The extraction was done as follows;

| STRAIN | SOLVENT | EXTRACTION METHOD |
|---|---|---|
| HBMSM 001 | chloroform/methanol/water | bead beating for 20 |
| | | minutes |
| HBMSM 002 | chloroform/methanol/water | bead beating for 20 |
| | | minutes |
| FBMSM 003 | hexane/methanol | bead beating for 20 |
| | | minutes |

| CODE | SAMPLE |
|---|---|
| M1 | HBMSM 001 |
| M2 | HBMSM 002 |
| M3 | HBMSM 003 |

The results are shown in FIG. 14 and Tables 2 and 3 hereinafter.

FIG. 14 shows the GC-MS for condition M3-N4. The identity of the peaks is given in Table 3 below.

As can be seen in the Tables, the overall system is dynamic. Table 2 shows how triacylglyceride-fatty acid metabolism responds to (stress) nitrogen limitation by a slight reduction in C16 TAG over 24 hours which recovers by 72 hours, and by increasing transiently C18 TAG production whilst reducing PUFA production. PUFA (Linolenic acid) increases over 72 hours.

Considering the indication at 12 hours nitrogen limitation, the bio-oil at that stage comprises approximately
23% C16 TAG
25% C18 TAG
9% C18 PUFA
4% C20 PUFA,
   with minor fractions of
carotenoid
sterol
chlorophyll.

The summary variation for the induced cells is as shown in Table 5 hereinafter.

### Example 10—Discussion of Sphaerica Triacylglyeride Metabolic Profile Compared with Freshwater Laboratory Model Organism Chlorella vulgaris

A comprehensive analysis of the triacylglyceride of the freshwater laboratory model organism Chlorella vulgaris has been reported by Pantami H A, Ahamad Bustamam M S, Lee S Y, Ismail I S, Mohd Faudzi S M, Nakakuni M, Shaari K. Comprehensive GCMS and LC-MS/MS Metabolite Profiling of Chlorella vulgaris. Mar Drugs. 2020 Jul. 17; 18 (7): 367. doi: 10.3390/md18070367. PMID: 32709006; PMCID: PMC7404257. Chlorella vulgaris is a laboratory model freshwater microalgal strain.

Compared to Chlorella, Sphaerica are distinguished by (1) the very high carbon flux to triacylglyceride allowing accumulation of e.g. up to 65% cell dry weight and; (2) the exact molecular profile of the triacylglyceride “bio-oil” which they produce discussed below.

Lipid content of the microalgae biomass in a photobioreactor and open pond was 2.26±0.51% and 3.18±0.80%, respectively. Fatty acid content ranged between 0.7-22.8% and 0.9-22.6% and the dominant fatty acids in both cultivating system was palmitic acid C16 (see Jay, M. I., M. Kawaroe, and H. Effendi. “Lipid and fatty acid composition microalgae Chlorella vulgaris using photobioreactor and open pond.” IOP Conference Series: Earth and Environmental Science. Vol. 141. No. 1. IOP Publishing, 2018).

In summary, according to published studies, Chlorella produces a low triacylglyceride content with an elaborate profile, showing some overlap in molecular identity with Sphaerica bio-oil.

In embodiments of the invention, Sphaerica bio-oil may optionally not comprise compounds more characteristic of Chlorella, or comprise compounds characteristic of Chlorella.

TABLE 3
see FIG. 14

| PK | RT | Library/ID | Area Pct |
|---|---|---|---|
| 50 | 33.905 | 9,12-Octadecadienoic acid (Z,Z)-, trimethylsilyl ester | 18.6136 |
| 39 | 31.4087 | Hexadecanoic acid, trimethylsilyl ester | 18.5183 |
| 51 | 33.9911 | .alpha.-Linolenic acid, trimethylsilyl ester | 9.579 |
| 22 | 27.9593 | Bicyclo[3.1.1]heptane, 2,6,6-trimethyl- | 8.8428 |
| | | (1.alpha.,2.beta.,5.alpha.)- | |
| 26 | 28.6848 | 1-Hexadecyne | 3.7314 |
| 34 | 30.6401 | 5,8,11,14,17-Eicosapentaenoic acid, methyl ester, (all- | |
| | | Z)- | 3.5906 |
| 114 | 47.6226 | .beta.-Sitosterol trimethylsilyl ether | 3.2569 |
| 47 | 33.2902 | Silane, [(3,7,11,15-tetramethyl-2- | 2.6038 |
| | | hexadecenyl)oxy]trimethyl- | |
| 35 | 30.8184 | 1,5-Cyclodecadiene, (E,Z)- | 2.4401 |
| 25 | 28.3651 | Dodeca-1,6-dien-12-ol, 6,10-dimethyl- | 2.4223 |
| 53 | 34.3539 | Octadecanoic acid, trimethylsilyl ester | 2.3167 |
| 52 | 34.0649 | trans-13-Octadecenoic acid, trimethylsilyl ester | 1.4753 |
| 30 | 29.7854 | n-Pentadecanoic acid, trimethylsilyl ester | 1.0036 |
| 38 | 31.2119 | Butanoic acid, 3-methyl-2-oxo-, trimethylsilyl ester | 0.9943 |
| 57 | 36.0755 | Arachidonic acid | 0.8038 |
| 23 | 28.0269 | Disparlure | 0.7511 |
| 36 | 30.9168 | Propanoic acid, 2-oxo-, trimethylsilyl ester | 0.7486 |
| 33 | 30.5479 | 9,12-Octadecadiynoic acid, trimethylsilyl ester | 0.6428 |
| 24 | 28.1253 | Tetradecanoic acid, trimethylsilyl ester | 0.6194 |
| 109 | 45.569 | 2-Ethylacridine | 0.5334 |
| 37 | 31.0213 | Heptadecanoic acid, trimethylsilyl ester | 0.5323 |
| 60 | 36.3768 | Linolenic acid, trimethylsilyl ester | 0.5108 |
| 45 | 32.8905 | Heptadecanoic acid, trimethylsilyl ester | 0.4657 |
| 108 | 45.3415 | Cholesterol trimethylsilyl ether | 0.459 |
| 40 | 32.1711 | Methyl 9-cis,11-trans-octadecadienoate | 0.4518 |
| 96 | 43.3555 | Di(1,2,3,5,6,7-hexahydro-pyrido[3.2.1-ij]quinolin-9- | 0.4502 |
| | | yl)disulfide | |
| 67 | 38.3443 | Diisooctyl phthalate | 0.4075 |
| 74 | 38.8301 | Hexanoic acid, 6-chloro-, trimethylsilyl ester | 0.4067 |
| 101 | 43.5891 | 1-{2-[3-(2-Acetyloxiran-2-yl)-1,1- | 0.4049 |
| | | dimethylpropyl]cycloprop-2-enyl}ethanone | |
| 113 | 46.5097 | Silane, [[(3.beta.,24R)-ergost-5-en-3-yl]oxy]trimethyl- | 0.3868 |
| 69 | 38.5596 | 1H-Indole, 5-methyl-2-phenyl- | 0.3838 |
| 29 | 29.4903 | Hexadecanoic acid, methyl ester | 0.3809 |
| 27 | 28.9246 | Bromoacetic acid, hexadecyl ester | 0.3724 |
| 75 | 38.9654 | Hexadecanoic acid, 2,3-bis[(trimethylsilyl)oxy]propyl | 0.3697 |
| | | ester | |
| 107 | 45.2861 | (+)-.alpha.-Tocopherol, O-trimethylsilyl- | 0.3606 |
| 41 | 32.2695 | 9,12,15-Octadecatrienoic acid, methyl ester, (Z,Z,Z)- | 0.3535 |
| 49 | 33.6529 | Arachidonic acid, trimethylsilyl ester | 0.3502 |
| 18 | 27.0063 | 12,12-Dimethoxydodecanoic acid, methyl ester | 0.3343 |
| 90 | 42.0335 | Tetracosanoic acid, trimethylsilyl ester | 0.3295 |
| 58 | 36.1616 | cis,cis,cis-7,10,13-Hexadecatrienal | 0.3249 |
| 61 | 36.4751 | cis-4,7,10,13,16,19-Docosahexaenoic acid, tert- | 0.3162 |
| | | butyldimethylsilyl ester | |
| 14 | 26.5144 | Phosphoric acid, bis(trimethylsilyl) 2,3- | 0.3047 |
| | | bis[(trimethylsilyl)oxy]propyl ester | |
| 28 | 29.1767 | Hexanoic acid, 6-iodo-, trimethylsilyl ester | 0.2871 |
| 56 | 35.559 | Hexadecanoic acid, trimethylsilyl ester | 0.277 |
| 42 | 32.5031 | Hexadecanoic acid, trimethylsilyl ester | 0.2654 |
| 12 | 25.4998 | Heptadecane | 0.2405 |
| 86 | 40.8591 | Hexanoic acid, 6-chloro-, trimethylsilyl ester | 0.2374 |
| 110 | 45.8334 | Vanadium, (.eta.7-cycloheptatrienylium)(.eta.5-2,4- | 0.2296 |
| | | cyclopentadien-1-yl)- | |
| 105 | 45.0341 | Melibiose, octakis(trimethylsilyl)- | 0.2287 |
| 63 | 36.8871 | Octadecanoic acid, trimethylsilyl ester | 0.2062 |
| 3 | 16.0678 | Glycerol, tris(trimethylsilyl) ether | 0.2053 |
| 6 | 20.7838 | 1H-Indole, 2,3,5-trimethyl-1-(trimethylsilyl)- | 0.1976 |
| 55 | 35.1778 | Glyceryl-glycoside TMS ether | 0.1951 |
| 87 | 40.9821 | 1-Monooleoylglycerol trimethylsilyl ether | 0.1741 |
| 21 | 27.8056 | 2-Hexadecene, 3,7,11,15-tetramethyl-, [R-[R*,R*-(E)]]- | 0.1739 |
| 64 | 37.0224 | 3,10-Dioxa-2,11-disiladodeca-5,7-diene, 2,2,11,11- | 0.1715 |
| | | tetramethyl- | |
| 8 | 22.1919 | Lycopodan-5-one, 12-hydroxy-15-methyl-, (15R)- | 0.165 |
| 20 | 27.5842 | 2-Cyclohexyldimethylsilyloxyoct-3-ene | 0.1618 |
| 43 | 32.5769 | Hexadecanoic acid, trimethylsilyl ester | 0.1616 |
| 11 | 25.383 | L-(−)-Sorbose, pentakis(trimethylsilyl) ether | 0.1581 |
| 48 | 33.5607 | Linolenic acid, trimethylsilyl ester | 0.1547 |
| 84 | 40.0844 | D-Galactose, 2,3,4,5,6-pentakis-O-(trimethylsilyl)- | 0.1474 |
| 112 | 46.2208 | Vanadium, (.eta.7-cycloheptatrienylium)(.eta.5-2,4- | 0.1472 |
| | | cyclopentadien-1-yl)- | |
| 99 | 43.4907 | 3-.alpha.-Mannobiose, octakis(trimethylsilyl) ether | 0.147 |
| | | (isomer 1) | |
| 79 | 39.654 | Docosanoic acid, trimethylsilyl ester | 0.1304 |
| 89 | 41.3326 | 1-Trimethylsilyl-3-(dimethyl-n-pentylsilyl)but-1-ene | 0.1288 |
| 103 | 44.7328 | Hexanoic acid, 6-chloro-, trimethylsilyl ester | 0.1241 |
| 88 | 41.0805 | .beta.-D-(−)-Ribopyranose, tetrakis(trimethylsilyl) ether | 0.116 |
| 80 | 39.6909 | ((((Carboxymethyl)thio)carbothioyl)thio)acetic acid | 0.1158 |
| | | ditbdms | |
| 62 | 36.6719 | 3-Methyl-1-di(tert-butyl)silyloxybutane | 0.1116 |
| 65 | 37.213 | (2R,3R)-(−)-2-Benzyloxy-1,3,4-butanetriol, | 0.1071 |
| | | tris(trimethylsilyl) ether | |
| 71 | 38.6395 | Silane, trimethyl(2-pentenyloxy)-, (Z)- | 0.102 |
| 16 | 26.6373 | 5-O-Methyl-d-gluconic acid dimethylamide | 0.1014 |
| 82 | 39.82 | Octadec-9Z-enol trimethylsilyl ether | 0.0978 |
| 102 | 44.4745 | Hexanoic acid, 6-chloro-, trimethylsilyl ester | 0.092 |
| 111 | 45.901 | 3′-Chlorooxanilic acid N′-(3-ethoxy-4- | 0.0919 |
| | | hydroxybenzylidene)hydrazide | |
| 1 | 12.0036 | 1-Pentamethyldisilyloxycyclopentane | 0.0906 |
| 2 | 15.4038 | Quinoline, 6-methoxy-, 1-oxide | 0.0904 |
| 54 | 34.6244 | 11-trans-Octadecenoic acid, trimethylsilyl ester | 0.0882 |
| 13 | 26.4283 | (But-2-enyloxy)trimethylsilane | 0.087 |
| 17 | 26.8771 | Silane, trimethyl(2-methylpropoxy)- | 0.0843 |
| 104 | 44.8434 | Vanadium, (.eta.7-cycloheptatrienylium)(.eta.5-2,4- | 0.0833 |
| | | cyclopentadien-1-yl)- | |
| 98 | 43.4477 | 1,2-Di(prop-2-enyl)-tetramethyldisilane | 0.0821 |
| 76 | 39.3404 | Aucubin, hexakis(trimethylsilyl) ether | 0.0788 |
| 73 | 38.7256 | 1-Di(tert-butyl)silyloxycyclopentane | 0.0784 |
| 106 | 45.1939 | Octasiloxane, 1,1,3,3,5,5,7,7,9,9,11,11,13,13,15,15- | 0.0775 |
| | | hexadecamethyl- | |
| 66 | 38.246 | .alpha.-D-Glucopyranoside, methyl 2-(acetylamino)-2- | 0.0768 |
| | | deoxy-3-O-(trimethylsilyl)-, cyclic methylboronate | |
| 31 | 30.2404 | 1-(t-Butyldimethylsilyl)-4-(2,2-dimethyl-6-methylene- | 0.0744 |
| | | cyclohexyl)-butan-1-ol | |
| 95 | 43.171 | 1-Trimethylsilyl-3-(dimethyl-n-pentylsilyl)but-1-ene | 0.0738 |
| 100 | 43.5277 | D-(+)-Galacturonic acid, O-pentakis(trimethylsilyl) deriv. | 0.0714 |
| 9 | 23.6552 | Benzothiazole, 2-phenyl- | 0.0684 |
| 46 | 33.0565 | 9,12-Octadecadienoic acid (Z,Z)- | 0.0659 |
| 77 | 39.365 | 2-Bromosebacic acid, bis(trimethylsilyl) ester | 0.063 |
| 4 | 16.2707 | Silanol, trimethyl-, phosphate (3:1) | 0.0614 |
| 70 | 38.6149 | Dodecahydropyrido[1,2-b]isoquinolin-6-one | 0.0585 |
| 19 | 27.4244 | Propanoic acid, pentamethyldisilanyl ester | 0.0552 |
| 59 | 36.2784 | 9,12-Octadecadiynoic acid, trimethylsilyl ester | 0.055 |
| 10 | 25.1125 | 8-Heptadecene | 0.0517 |
| 97 | 43.4047 | Dihydrofuranno(3,2-H)homochromanone | 0.0506 |
| 15 | 26.5758 | beta.-D-Fructofuranose, 2,3,4,6-tetrakis-O- | 0.0489 |
| | | (trimethylsilyl)-, bis(trimethylsilyl) phosphate | |
| 44 | 32.6814 | 1-Butyldimethylsilyloxy-3-methylbut-2-ene | 0.0486 |
| 78 | 39.5433 | Azelaic acid, bis(trimethylsilyl) ester | 0.0486 |
| 91 | 42.4824 | trans-13-Octadecenoic acid, trimethylsilyl ester | 0.0461 |
| 72 | 38.701 | 17-Octadecynoic acid, tert-butyldimethylsilyl ester | 0.0454 |
| 68 | 38.4796 | trans-Crotyl alcohol, trimethylsilyl ether | 0.0385 |
| 7 | 21.0421 | 3-Amino-2-[2,2-bis(methyloxy)ethyl]benzonitrile | 0.0356 |
| 32 | 30.2589 | 2-Oxiranemethanol, .alpha.-(1-methylethyl)-3-[1- | 0.0337 |
| | | (trimethylsilyloxy)pentyl]- | |
| 92 | 42.6054 | Hexanoic acid, 6-chloro-, trimethylsilyl ester | 0.031 |
| 83 | 39.8999 | cis-13-Octadecenoic acid, trimethylsilyl ester | 0.0285 |
| 85 | 40.5701 | .alpha.-D-Glucopyranoside, methyl 2-(acetylamino)-2- | 0.0273 |
| | | deoxy-3-O-(trimethylsilyl)-, cyclic methylboronate | |
| 81 | 39.7585 | Silane, trimethyl[(4-methylcyclohexyl)oxy]- | 0.0269 |
| 94 | 43.1157 | trans-Crotyl alcohol, tert-butyldimethylsilyl ether | 0.0251 |
| 93 | 43.0419 | Hexanoic acid, 6-chloro-, trimethylsilyl ester | 0.0154 |
| 115 | 47.764 | Thiocarbamic acid, N,N-dimethyl, S-1,3-diphenyl-2- | 0.0112 |
| | | butenyl ester | |
| 117 | 50.0637 | Vanadium, (.eta.7-cycloheptatrienylium)(.eta.5-2,4- | 0.0108 |
| | | cyclopentadien-1-yl)- | |
| 5 | 16.3384 | Silanol, trimethyl-, phosphate (3:1) | 0.008 |
| 118 | 50.0944 | Hexadecandioic acid, bis(trimethylsilyl) ester | 0.0049 |
| 116 | 47.801 | 4-Dehydroxy-N-(4,5-methylenedioxy-2- | 0.0034 |
| | | nitrobenzylidene)tyramine | |

### GENERAL REFERENCES

1. Guiry, M. D. (2012), How many species of algae are there? J. Phycol. 48:1057-1067 https://doi.org/10.1111/j. 1529-8817.2012.01222.x.
2. Mora, C., Tittensor, D. P., Adl, S., Simpson, A. G. B. & Worm, B. 2011. How many species are there on earth and in the ocean? PLOS Biol. 9: e1001127. doi: 10.1371/journal. pbio. 1001127.
3. Rodolfi L, Zittelli G C, Bassi N, Padovani G, Biondi N, Bonini G, et al. Microalgae for oil: strain selection, induction of lipid synthesis and outdoor mass cultivation in a low-cost photobioreactor. Biotechnol Bioeng. 2009; 102:100-12.
4. Sydney, E. B., da Silva, T. E., Tokarski, A., Novak, A. C., de Carvalho, J. C., Woiciecohwski, A. L., Larroche, C., & Soccol, C. R. (2011). Screening of microalgae with potential for biodiesel production and nutrient removal from treated domestic sewage. Applied Energy, 88 (10), 3291-3294. https://doi.org/10.1016/j.apenergy.2010.11.024.
5. Benedetti, M., Vecchi, V., Barera, S., & Dall'Osto, L. (2018). Biomass from microalgae: the potential of domestication towards sustainable biofactories. Microbial Cell Factories, 17 (1), 173. https://doi.org/10.1186/s12934-018-1019-3).
6. Nascimento, I. A., Marques, S. S. I., Cabanelas, I. T. D., Pereira, S. A., Druzian, J. I., de Souza, C. O., Vich, D. V., de Carvalho, G. C., & Nascimento, M. A. (2013). Screening Microalgae Strains for Biodiesel Production: Lipid Productivity and Estimation of Fuel Quality Based on Fatty Acids Profiles as Selective Criteria. Bioenergy Research, 6 (1), 1-13. https://doi.org/10.1007/s12155-012-9222-2.
7. Dahlin, L. R., Van Wychen, S., Gerken, H. G., McGowen, J., Pienkos, P. T., Posewitz, M. C., & Guarnieri, M. T. (2018). Down-Selection and Outdoor Evaluation of Novel, Halotolerant Algal Strains for Winter Cultivation. Frontiers in Plant Science, 9, 1513. https://doi.org/10.3389/fpls.2018.01513.
8. Davis R, Markham J, Kinchin C M, Grundl N, Tan, E C D, Humbird, D. (2016). Process Design and Economics for the Production in Open Pond Systems and Processing Through Dewatering for Downstream Conversion. National Renewable Energy Laboratory, February, 128. https://doi.org/10.2172/1239893.
9. Davis, R., & Laurens, L. (2018). Algal Biomass Production via Open Pond Algae Farm Cultivation: 2019 State of Technology and Future Research. www.nrel.gov/publications.
10. Melis A. Solar energy conversion efficiencies in photosynthesis: minimizing the chlorophyll antennae to maximize efficiency. Plant Sci. 2009; 177:272-80. doi: 10.1016/j.plantsci.2009.06.005.
11. Zou, N., & Richmond, A. (2000). Light-path length and population density in photoacclimation of Nannochloropsis sp. (Eustigmatophyceae). Journal of Applied Phycology, 12 (3-5), 349-354. https://doi.org/10.1023/a: 1008151004317.
12. Borowitzka, M. A. (2005). The Mass Culture of Dunaliella salina. In Technical Resource Papers Regional Workshop on the Culture and Utilization of Seaweeds Volume II. http://www.fao.org/3/AB728E/AB728E06.htm.
13. Huesemann, M. H., Hausmann, T. S., Bartha, R., Aksoy, M., Weissman, J. C., & Benemann, J. R. (2009). Biomass productivities in wild type and pigment mutant of cyclotella sp. (Diatom). Applied Biochemistry and Biotechnology, 157 (3), 507-526. https://doi.org/10.1007/s12010-008-8298-9.
14. Smith, V. H., & Crews, T. (2014). Applying ecological principles of crop cultivation in large-scale algal biomass production. Algal Research, 4 (1), 23-34. https://doi.org/10.1016/j.algal.2013.11.005.
15. Smith, V. H., Foster, B. L., Grover, J. P., Holt, R. D., Leibold, M. A., & DeNoyelles, F. (2005). Phytoplankton species richness scales consistently from laboratory microcosms to the world's oceans. Proceedings of the National Academy of Sciences of the United States of America, 102 (12), 4393-4396. https://doi.org/10.1073/pnas.0500094102.
16. Schroeder, D. C., Oke, J., Hall, M., Malin, G., & Wilson, W. H. (2003). Virus succession observed during an Emiliania huxleyi bloom. Applied and Environmental Microbiology, 69 (5), 2484-2490. https://doi.org/10.1128/AEM.69.5.2484-2490.2003.
17. Gutman, J., Zarka, A., & Boussiba, S. (2009). The host-range of Paraphysoderma sedebokerensis, a chytrid that infects Haematococcus pluvialis. European Journal of Phycology, 44 (4), 509-514. https://doi.org/10.1080/09670260903161024.
18. Strittmatter, M., Guerra, T., Silva, J., & Gachon, C. M. M. (2016). A new flagellated dispersion stage in Paraphysoderma sedebokerense, a pathogen of Haematococcus pluvialis. Journal of Applied Phycology, 28 (3), 1553-1558. https://doi.org/10.1007/s10811-015-0700-8.
19. Day, J. G., Thomas, N. J., Achilles-Day, U. E. M., & Leakey, R. J. G. (2012). Early detection of protozoan grazers in algal biofuel cultures. Bioresource Technology, 114, 715-719. https://doi.org/10.1016/j.biortech.2012.03.015.
20. Carvalho, A. P., Silva, S. O., Baptista, J. M., & Malcata, F. X. (2011). Light requirements in microalgal photobioreactors: An overview of biophotonic aspects. In Applied Microbiology and Biotechnology (Vol. 89, Issue 5, pp. 1275-1288). Springer. https://doi.org/10.1007/s00253-010-3047-8.
21. Rochaix, J.-D. (2014). Regulation and Dynamics of the Light-Harvesting System. Annual Review of Plant Biology, 65 (1), 287-309. https://doi.org/10.1146/annurev-arplant-050213-040226.
22. Barber, J. (2006). Photosystem II: An enzyme of global significance. Biochemical Society Transactions, 34 (5), 619-631. https://doi.org/10.1042/BST0340619.
23. Blankenship, R. E. (2010). Early Evolution of Photosynthesis. Plant Physiology, 154 (2), 434-438. https://doi.org/10.1104/PP.110.161687.
24. Wientjes, E., Van Amerongen, H., & Croce, R. (2013). LHCII is an antenna of both photosystems after long-term acclimation. Biochimica et Biophysica Acta-Bioenergetics, 1827 (3), 420-426. https://doi.org/10.1016/j.bbabio.2012.12.009.
25. Rochaix, J. D., Lemeille, S., Shapiguzov, A., Samol, I., Fucile, G., Willig, A., & Goldschmidt-Clermont, M. (2012). Protein kinases and phosphatases involved in the acclimation of the photosynthetic apparatus to a changing light environment. In Philosophical Transactions of the Royal Society B: Biological Sciences (Vol. 367, Issue 1608, pp. 3466-3474). Royal Society. https://doi.org/10.1098/rstb.2012.0064.
26. Niyogi, K. K. (1999). Photoprotection revisited: Genetic and molecular approaches. Annual Review of Plant Biology, 50, 333-359. https://doi.org/10.1146/annurev.arplant.50.1.333.
27. Girolomoni, L., Cazzaniga, S., Pinnola, A., Perozeni, F., Ballottari, M., & Bassi, R. (2019). LHCSR3 is a nonphotochemical quencher of both photosystems in Chlamydomonas reinhardtii. Proceedings of the National Academy of Sciences of the United States of America, 116 (10), 4212-4217. https://doi.org/10.1073/pnas. 1809812116.
28. Tian, L., Nawrocki, W. J., Liu, X., Polukhina, I., Van Stokkum, I. H. M., & Croce, R. (2019). PH dependence, kinetics and light-harvesting regulation of nonphotochemical quenching in Chlamydomonas. Proceedings of the National Academy of Sciences of the United States of America, 116 (17), 8320-8325. https://doi.org/10.1073/pnas.1817796116.
29. Rochaix, J. D., & Bassi, R. (2019). LHC-like proteins involved in stress responses and biogenesis/repair of the photosynthetic apparatus. In Biochemical Journal (Vol. 476, Issue 3, pp. 581-593). Portland Press Ltd. https://doi.org/10.1042/BCJ20180718.
30. Pinnola, A., & Griffiths, H. (2019). The rise and fall of Light-Harvesting Complex Stress-Related proteins as photoprotection agents during evolution. In Journal of Experimental Botany (Vol. 70, Issue 20, pp. 5527-5535). Oxford University Press. https://doi.org/10.1093/jxb/erz317.
31. Ananyev, G., Gates, C., Kaplan, A., & Dismukes, G. C. (2017). Photosystem II-cyclic electron flow powers exceptional photoprotection and record growth in the microalga Chlorella ohadii. Biochimica et Biophysica Acta-Bioenergetics, 1858 (11), 873-883. https://doi.org/10.1016/j.bbabio.2017.07.001.
32. Friedland, N., Negi, S., Vinogradova-Shah, T., Wu, G., Ma, L., Flynn, S., Kumssa, T., Lee, C. H., & Sayre, R. T. (2019). Fine-tuning the photosynthetic light harvesting apparatus for improved photosynthetic efficiency and biomass yield. Scientific Reports, 9 (1), 1-12. https://doi.org/10.1038/s41598-019-49545-8.
33. Kolodny, Y., Zer, H., Propper, M., Yochelis, S., Paltiel, Y., & Keren, N. (2021). Marine cyanobacteria tune energy transfer efficiency in their light-harvesting antennae by modifying pigment coupling. FEBS Journal, 288 (3), 980-994. https://doi.org/10.1111/febs.15371.
34. Rochaix, J. D., & Bassi, R. (2019). LHC-like proteins involved in stress responses and biogenesis/repair of the photosynthetic apparatus. In Biochemical Journal (Vol. 476, Issue 3, pp. 581-593).
35. Tikkanen, M., Mekala, N. R., & Aro, E. M. (2014). Photosystem II photoinhibition-repair cycle protects Photosystem i from irreversible damage. Biochimica et Biophysica Acta-Bioenergetics, 1837 (1), 210-215. https://doi.org/10.1016/j.bbabio.2013.10.001).
36. Bassi, R., & Caffarri, S. (2000). Lhc proteins and the regulation of photosynthetic light harvesting function by xanthophylls. In Photosynthesis Research (Vol. 64).
37. Vecchi, V., Barera, S., Bassi, R., & Dall'osto, L. (2020). Potential and challenges of improving photosynthesis in algae. In Plants (Vol. 9, Issue 1, p. 67). MDPI AG. https://doi.org/10.3390/plants9010067.
38. Tian, L., Nawrocki, W. J., Liu, X., Polukhina, I., Van Stokkum, I. H. M., & Croce, R. (2019). PH dependence, kinetics and light-harvesting regulation of nonphotochemical quenching in Chlamydomonas. Proceedings of the National Academy of Sciences of the United States of America, 116 (17), 8320-8325. https://doi.org/10.1073/pnas. 1817796116.
39. Li, L., Aro, E. M., & Millar, A. H. (2018). Mechanisms of Photodamage and Protein Turnover in Photoinhibition. In Trends in Plant Science (Vol. 23, Issue 8, pp. 667-676). Elsevier Ltd. https://doi.org/10.1016/j.tplants.2018.05.004.
40. Wang, B., Lan, C. Q., & Horsman, M. (2012). Closed photobioreactors for production of microalgal biomasses. In Biotechnology Advances (Vol. 30, Issue 4, pp. 904-912). Elsevier. https://doi.org/10.1016/j.biotechadv.2012.01.019.
41. Davis, R., & Laurens, L. (2020). Algal Biomass Production via Open Pond Algae Farm Cultivation: 2019 State of Technology and Future Research. https://www.nrel.gov/docs/fy20osti/76569.pdf.
42. Tanaka, Y., Nishiyama, Y., & Murata, N. (2000). Acclimation of the photosynthetic machinery to high temperature in Chlamydomonas reinhardtii requires synthesis de novo of proteins encoded by the nuclear and chloroplast genomes. Plant Physiology, 124 (1), 441-449. https://doi.org/10.1104/pp. 124.1.441.
43. Kobayashi, Y., Harada, N., Nishimura, Y., Saito, T., Nakamura, M., Fujiwara, T., Kuroiwa, T., & Misumi, O. (2014). Algae sense exact temperatures: Small heat shock proteins are expressed at the survival threshold temperature in cyanidioschyzon merolae anfted Chlamydomonas reinhardtii. Genome Biology and Evolution, 6 (10), 2731-2740. https://doi.org/10.1093/gbe/evu216.
44. Allen, J. W., Tevatia, R., Demirel, Y., DiRusso, C. C., & Black, P. N. (2018). Induction of oil accumulation by heat stress is metabolically distinct from N stress in the green microalgae Coccomyxa subellipsoidea C169. PLOS ONE, 13 (9), e0204505. https://doi.org/10.1371/journal.pone.0204505.
45. Hemme, D., Veyel, D., Mühlhaus, T., Sommer, F., Jüppner, J., Unger, A. K., Sandmann, M., Fehrle, I., Schönfelder, S., Steup, M., Geimer, S., Kopka, J., Giavalisco, P., & Schroda, M. (2014). Systems-Wide analysis of acclimation responses to long-term heat stress and recovery in the photosynthetic model organism Chlamydomonas reinhardtii. Plant Cell, 26 (11), 4270-4297. https://doi.org/10.1105/tpc.114.130997.
46. Schmollinger, S., Schulz-Raffelt, M., Strenkert, D., Veyel, D., Vallon, O., & Schroda, M. (2013). Dissecting the heat stress response in chlamydomonas by pharmaceutical and RNAi approaches reveals conserved and novel aspects. Molecular Plant, 6 (6), 1795-1813. https://doi.org/10.1093/mp/sst086.
47. Lyon, B., & Mock, T. (2014). Polar Microalgae: New Approaches towards Understanding Adaptations to an Extreme and Changing Environment. Biology, 3 (1), 56-80. https://doi.org/10.3390/biology3010056.
48. Sakamoto, T., & Murata, N. (2002). Regulation of the desaturation of fatty acids and its role in tolerance to cold and salt stress. In Current Opinion in Microbiology (Vol. 5, Issue 2, pp. 206-210). Elsevier Ltd. https://doi.org/10.1016/S1369-5274 (02) 00306-5.
49. Jensen, R. G. (2000). Activation of Rubisco regulates photosynthesis at high temperature and CO2. In Proceedings of the National Academy of Sciences of the United States of America (Vol. 97, Issue 24, pp. 12937-12938). National Academy of Sciences. https://doi.org/10.1073/pnas.97.24.12937.
50. Cakmak, T., Angun, P., Demiray, Y. E., Ozkan, A. D., Elibol, Z., & Tekinay, T. (2012). Differential effects of nitrogen and sulfur deprivation on growth and biodiesel feedstock production of Chlamydomonas reinhardtii. Biotechnology and Bioengineering, 109 (8), 1947-1957. https://doi.org/10.1002/bit.24474.
51. Li, X., Moellering, E. R., Liu, B., Johnny, C., Fedewa, M., Sears, B. B., Kuo, M.-H., & Benning, C. (2012). A Galactoglycerolipid Lipase Is Required for Triacylglycerol Accumulation and Survival Following Nitrogen Deprivation in Chlamydomonas reinhardtii. The Plant Cell, 24 (11), 4670-4686. https://doi.org/10.1105/tpc. 112.105106.
52. Schmollinger, S., Mühlhaus, T., Boyle, N. R., Blaby, I. K., Casero, D., Mettler, T., Moseley, J. L., Kropat, J., Sommer, F., Strenkert, D., Hemme, D., Pellegrini, M., Grossman, A. R., Stitt, M., Schroda, M., & Merchant, S. S. (2014). Nitrogen-sparing mechanisms in Chlamydomonas affect the transcriptome, the proteome, and photosynthetic metabolism. Plant Cell, 26 (4), 1410-1435. https://doi.org/10.1105/tpc.113.122523.
53. Hema, R., Senthil-Kumar, M., Shivakumar, S., Chandrasekhara Reddy, P., & Udayakumar, M. (2007). Chlamydomonas reinhardtii, a model system for functional validation of abiotic stress responsive genes. Planta, 226 (3), 655-670. https://doi.org/10.1007/s00425-007-0514-2.
54. Khona, D. K., Shirolikar, S. M., Gawde, K. K., Hom, E., Deodhar, M. A., & D'Souza, J. S. (2016). Characterization of salt stress-induced palmelloids in the green alga, Chlamydomonas reinhardtii. Algal Research, 16, 434-448. https://doi.org/10.1016/j.algal.2016.03.035.
55. Allakhverdiev, S. I., Nishiyama, Y., Suzuki, I., Tasaka, Y., & Murata, N. (1999). Genetic engineering of the unsaturation of fatty acids in membrane lipids alters the tolerance of Synechocystis to salt stress. Proceedings of the National Academy of Sciences of the United States of America, 96 (10), 5862-5867. https://doi.org/10.1073/pnas.96.10.5862.
56. Allakhverdiev, S. I., & Murata, N. (2008). Salt stress inhibits photosystems II and I in cyanobacteria. In Photosynthesis Research (Vol. 98, Issues 1-3, pp. 529-539). Photosynth Res. https://doi.org/10.1007/s11120-008-9334-x.
57. Allakhverdiev, S. I., Kinoshita, M., Inaba, M., Suzuki, I., & Murata, N. (2001). Unsaturated fatty acids in membrane lipids protect the photosynthetic machinery against salt-induced damage in Synechococcus. Plant Physiology, 125 (4), 1842-1853. https://doi.org/10.1104/pp. 125.4.1842.
58. Ahmad, I., & Hellebust, J. A. (1984). Osmoregulation in the Extremely Euryhaline Marine Micro-Alga Chlorella autotrophica. Plant Physiology, 74 (4), 1010-1015. https://doi.org/10.1104/pp. 74.4.1010.
59. Hellebust, J. A., & Le Gresley, S. M. L. (1985). Growth characteristics of the marine rock pool flagellate Chlamydomonas pulsatilla Wollenweber (Chlorophyta). Phycologia, 24 (2), 225-229. https://doi.org/10.2216/i0031-8884-24-2-225.1.
60. Ho, S. H., Nakanishi, A., Kato, Y., Yamasaki, H., Chang, J. S., Misawa, N., Hirose, Y., Minagawa, J., Hasunuma, T., & Kondo, A. (2017). Dynamic metabolic profiling together with transcription analysis reveals salinity-induced starch-To-lipid biosynthesis in alga Chlamydomonas sp. JSC4. Scientific Reports, 7. https://doi.org/10.1038/srep45471.
61. Salama, E. S., Kim, H. C., Abou-Shanab, R. A. I., Ji, M. K., Oh, Y. K., Kim, S. H., & Jeon, B. H. (2013). Biomass, lipid content, and fatty acid composition of freshwater Chlamydomonas mexicana and Scenedesmus obliquus grown under salt stress. Bioprocess and Biosystems Engineering, 36 (6), 827-833. https://doi.org/10.1007/s00449-013-0919-1.
62. Bremauntz, M. del P., Torres-Bustillos, L. G., Cañizares-Villanueva, R.-O., Duran-Paramo, E., & Fernández-Linares, L. (2011). Trehalose and Sucrose Osmolytes Accumulated by Algae as Potential Raw Material for Bioethanol. Natural Resources, 02 (03), 173-179. https://doi.org/10.4236/nr.2011.23023.
63. Brown, L. M., & Hellebust, J. A. (1978). Sorbitol and proline as intracellular osmotic solutes in the green alga Stichococcus bacillaris. Canadian Journal of Botany, 56 (6), 676-679. https://doi.org/10.1139/b78-074.
64. Smith, V. H., Foster, B. L., Grover, J. P., Holt, R. D., Leibold, M. A., & DeNoyelles, F. (2005). Phytoplankton species richness scales consistently from laboratory microcosms to the world's oceans. Proceedings of the National Academy of Sciences of the United States of America, 102 (12), 4393-4396. https://doi.org/10.1073/pnas.0500094102.
65. Schroeder, D. C., Oke, J., Hall, M., Malin, G., & Wilson, W. H. (2003). Virus succession observed during an Emiliania huxleyi bloom. Applied and Environmental Microbiology, 69 (5), 2484-2490. https://doi.org/10.1128/AEM.69.5.2484-2490.2003.
66. Gutman, J., Zarka, A., & Boussiba, S. (2009). The host-range of Paraphysoderma sedebokerensis, a chytrid that infects Haematococcus pluvialis. European Journal of Phycology, 44 (4), 509-514. https://doi.org/10.1080/09670260903161024.
67. Strittmatter, M., Guerra, T., Silva, J., & Gachon, C. M. M. (2016). A new flagellated dispersion stage in Paraphysoderma sedebokerense, a pathogen of Haematococcus pluvialis. Journal of Applied Phycology, 28 (3), 1553-1558. https://doi.org/10.1007/s10811-015-0700-8.
68. Day, J. G., Thomas, N. J., Achilles-Day, U. E. M., & Leakey, R. J. G. (2012). Early detection of protozoan grazers in algal biofuel cultures. Bioresource Technology, 114, 715-719. https://doi.org/10.1016/j.biortech.2012.03.015.
69. Hingamp, P., Grimsley, N., Acinas, S. G., Clerissi, C., Subirana, L., Poulain, J., Ferrera, I., Sarmento, H., Villar, E., Lima-Mendez, G., Faust, K., Sunagawa, S., Claverie, J. M., Moreau, H., Desdevises, Y., Bork, P., Raes, J., De Vargas, C., Karsenti, E., . . . . Ogata, H. (2013). Exploring nucleo-cytoplasmic large DNA viruses in Tara Oceans microbial metagenomes. ISME Journal, 7 (9), 1678-1695. https://doi.org/10.1038/ismej.2013.59.
70. Bratbak G, Egge J K, Heldal M. Viral mortality of the marine alga Emiliania huxleyi (Haptophyceae) and termination of algal blooms. Mar Ecol-Prog Ser. 1993; 93:39-48. 10.3354/meps093039.
71. Brussaard C P D, Kuipers B, Veldhuis M J W. A mesocosm study of Phaeocystis globosa population dynamics—1. Regulatory role of viruses in bloom. Harmful Algae. 2005; 4:859-874.
72. Dunigan, D. D., Fitzgerald, L. A., & Van Etten, J. L. (2006). Phycodnaviruses: A peek at genetic diversity. Virus Research, 117 (1), 119-132. https://doi.org/10.1016/j.virusres.2006.01.024.
73. Yau, S., Krasovec, M., Felipe Benites, L., Rombauts, S., Groussin, M., Vancaester, E., Aury, J. M., Derelle, E., Desdevises, Y., Escande, M. L., Grimsley, N., Guy, J., Moreau, H., Sanchez-Brosseau, S., van de Peer, Y., Vandepoele, K., Gourbiere, S., & Piganeau, G. (2020). Virus-host coexistence in phytoplankton through the genomic lens. Science Advances, 6 (14), eaay2587. https://doi.org/10.1126/sciadv.aay2587.
74. Thyrhaug, R., Larsen, A., Thingstad, T. F., & Bratbak, G. (2003). Stable coexistence in marine algal host-virus systems. Marine Ecology Progress Series, 254, 27-35. https://doi.org/10.3354/meps254027.
75. Van Etten, J. L., Lane, L. C., & Meints, R. H. (1991). Viruses and viruslike particles of eukaryotic algae. Microbiology and Molecular Biology Reviews, 55 (4).
76. Frickel, J., Sieber, M., & Becks, L. (2016). Eco-evolutionary dynamics in a coevolving host-virus system. Ecology Letters, 19 (4), 450-459. https://doi.org/10.1111/ele. 12580.
77. Thomas, R., Grimsley, N., Escande, M., Subirana, L., Derelle, E., & Moreau, H. (2011). Acquisition and maintenance of resistance to viruses in eukaryotic phytoplankton populations. Environmental Microbiology, 13 (6), 1412-1420. https://doi.org/10.1111/j. 1462-2920.2011.02441.x.
78. Yau, S., Hemon, C., Derelle, E., Moreau, H., Piganeau, G., & Grimsley, N. (2016). A Viral Immunity Chromosome in the Marine Picoeukaryote, Ostreococcus tauri. PLOS Pathogens, 12 (10). https://doi.org/10.1371/journal.ppat. 1005965.
79. De-Bashan, L. E., Trejo, A., Huss, V. A. R., Hernandez, J.-P., & Bashan, Y. (2007). Chlorella sorokiniana UTEX 2805, a heat and intense, sunlight-tolerant microalga with potential for removing ammonium from wastewater. https://doi.org/10.1016/j.biortech.2007.09.065.
80. Treves, H., Raanan, H., Finkel, O. M., Berkowicz, S. M., Keren, N., Shotland, Y., & Kaplan, A. (2013). A newly isolated Chlorella sp. from desert sand crusts exhibits a unique resistance to excess light intensity. FEMS Microbiology Ecology, 86 (3), 373-380. https://doi.org/10.1111/1574-6941.12162.
81. Tan, C. K., Ishizaka, J., Matsumura, S., Yusoff, F. M., & Mohamed, M. I. H. (2006). Seasonal variability of SeaWiFS chlorophyll a in the Malacca Straits in relation to Asian monsoon. Continental Shelf Research, 26 (2), 168-178. https://doi.org/10.1016/J.CSR.2005.09.008.
82. Schroeder, D. C., Oke, J., Hall, M., Malin, G., & Wilson, W. H. (2003). Virus succession observed during an Emiliania huxleyi bloom. Applied and Environmental Microbiology, 69 (5), 2484-2490. https://doi.org/10.1128/AEM.69.5.2484-2490.2003.
83. Hanic, L. A., Sekimoto, S., & Bates, S. S. (2009). Oomycete and chytrid infections of the marine diatom Pseudo-nitzschia pungens (Bacillariophyceae) from Prince Edward Island, Canada. Botany, 87 (11), 1096-1105. https://doi.org/10.1139/B09-070.
84. Mitra, A., & Flynn, K. J. (2006). Promotion of harmful algal blooms by zooplankton predatory activity. Biology Letters, 2 (2), 194-197. https://doi.org/10.1098/rsbl.2006.0447.
85. Han, D., Li, Y., and Hu, Q. (2013). “Biology and Commercial Aspects of Haematococcus pluvialis,” in Handbook of Microalgal Culture (Oxford, UK: John Wiley & Sons, Ltd), 388-405. doi: 10.1002/9781118567166.ch20.
86. Carney, L. T., and Lane, T. W. (2014). Parasites in algae mass culture. Front. Microbiol. 5, 278. doi: 10.3389/fmicb.2014.00278.
87. McBride, R., Smith, V. H., Carney, L. T., and Lane, T. W. (2016). “Crop Protection in Open Ponds,” in Microalgal Production for Biomass and High-Value Products (CRC Press), 139-163. doi: 10.1201/b19464-7.
88. Engel, B. D., Schaffer, M., Cuellar, L. K., Villa, E., Plitzko, J. M., & Baumeister, W. (2015). Native architecture of the chlamydomonas chloroplast revealed by in situ cryo-electron tomography. ELife, 2015 (4). https://doi.org/10.7554/eLife.04889.
89. Karlsson, J., Ciarke, A. K., Chen, Z. Y., Hugghins, S. Y., Park, Y. II, Husic, H. D., Moroney, J. V., & Samuelsson, G. (1998). A novel a-type carbonic anhydrase associated with the thylakoid membrane in Chlamydomonas reinhardtii is required for growth at ambient CO2. EMBO Journal, 17 (5), 1208-1216. https://doi.org/10.1093/emboj/17.5.1208.
90. Mckay, R. M. L., & Gibbs, S. P. (1991). Composition and function of pyrenoids: cytochemical and immunocytochemical approaches. Canadian Journal of Botany, 69 (5), 1040-1052. https://doi.org/10.1139/b91-134.
91. Wang, Y., Stessman, D. J., & Spalding, M. H. (2015). The CO2 concentrating mechanism and photosynthetic carbon assimilation in limiting CO2: How Chlamydomonas works against the gradient. Plant Journal, 82 (3), 429-448. https://doi.org/10.1111/tpj. 12829.
92. Moroney, J. V., & Ynalvez, R. A. (2007). Proposed carbon dioxide concentrating mechanism in Chlamydomonas reinhardtii. In Eukaryotic Cell (Vol. 6, Issue 8, pp. 1251-1259). American Society for Microbiology Journals. https://doi.org/10.1128/EC.00064-07.
93. Wang, Y., Duanmu, D., & Spalding, M. H. (2011). Carbon dioxide concentrating mechanism in Chlamydomonas reinhardtii: Inorganic carbon transport and CO2 recapture. Photosynthesis Research, 109 (1-3), 115-122. https://doi.org/10.1007/s11120-011-9643-3.
94. Cocquyt, E., Verbruggen, H., Leliaert, F., & De Clerck, O. (2010). Evolution and cytological diversification of the green seaweeds (Ulvophyceae). Molecular Biology and Evolution, 27 (9), 2052-2061. https://doi.org/10.1093/molbev/msq091.
95. Fučíková, K., Leliaert, F., Cooper, E. D., Škaloud, P., D'Hondt, S., De Clerck, O., Gurgel, C. F. D., Lewis, L. A., Lewis, P. O., Lopez-Bautista, J. M., Delwiche, C. F., & Verbruggen, H. (2014). New phylogenetic hypotheses for the core Chlorophyta based on chloroplast sequence data. Frontiers in Ecology and Evolution, 2 (OCT), 63. https://doi.org/10.3389/fevo.2014.00063.
96. Wetherbee, R., & Verbruggen, H. (2016). Kraftionema allantoideum, a new genus and family of Ulotrichales (Chlorophyta) adapted for survival in high intertidal pools. Journal of Phycology, 52 (5), 704-715. https://doi.org/10.1111/jpy.12447.
97. Thompson J D, Higgins D G, Gibson T J. (1994). CLUSTAL W: improving the sensitivity of progressive multiple sequence alignment through sequence weighting, position-specific gap penalties and weight matrix choice. Nucleic Acids Res., 22, 4673-4680.
98. Larkin M A, Blackshields G, Brown N P, Chenna R, McGettigan P A, McWilliam H, Valentin F, Wallace I M, Wilm A, Lopez R, Thompson J D, Gibson T J, Higgins D G. (2007). Clustal W and Clustal X version 2.0. Bioinformatics, 23, 2947-2948.
99. Saitou N., Nei M.: The Neighbor-Joining Method: a new method for reconstructing phylogenetic trees. Molecular Biology and Evolution, vol 4, pp. 406-425, 1987.
100. Wigmosta, M. S., Coleman, A. M., Skaggs, R. J., Huesemann, M. H., & Lane, L. J. (2011). National microalgae biofuel production potential and resource demand. Water Resources Research, 47 (4), 0-04. https://doi.org/10.1029/2010WR009966.
101. Venteris, E. R., Skaggs, R. L., Coleman, A. M., & Wigmosta, M. S. (2013). A GIS cost model to assess the availability of freshwater, seawater, and saline groundwater for algal biofuel production in the United States. Environmental Science and Technology, 47 (9), 4840-4849. https://doi.org/10.1021/es304135b.
102. Venteris, E. R., McBride, R. C., Coleman, A. M., Skaggs, R. L., & Wigmosta, M. S. (2014). Siting algae cultivation facilities for biofuel production in the united states: Trade-offs between growth rate, site constructability, water availability, and infrastructure. Environmental Science and Technology, 48 (6), 3559-3566. https://doi.org/10.1021/es4045488.
103. Xu, H., Lee, U., Coleman, A. M., Wigmosta, M. S., & Wang, M. (2019). Assessment of algal biofuel resource potential in the United States with consideration of regional water stress. Algal Research, 37, 30-39. https://doi.org/10.1016/j.algal.2018.11.002.
104. Davis, R., Markham, J., Kinchin, C., Zhu, Y., Jones, S., Han, J., Canter, C., & Li, Q. (2018). 2017 Algae Harmonization Study: Evaluating the Potential for Future Algal Biofuel Costs, Sustainability, and Resource Assessment from Harmonized Modeling Contributing Authors Algae Farm TEA: CAP Conversion TEA: HTL Conversion TEA: System LCA. https://www.nrel.gov/docs/fyl8osti/70715.pdf.
105. Yang, J., Xu, M., Zhang, X., Hu, Q., Sommerfeld, M., & Chen, Y. (2011). Life-cycle analysis on biodiesel production from microalgae: Water footprint and nutrients balance. Bioresource Technology, 102 (1), 159-165. https://doi.org/10.1016/j.biortech.2010.07.017.
106. Committee on the Sustainable Development of Algal Biofuels, National Research Council of the National Academies, (2012), Sustainable Development of Algal Biofuels, The Natl. Acad. Press, Washington, D.C.
107. Bloom, J. D., & Arnold, F. H. (2009). In the light of directed evolution: pathways of adaptive protein evolution. In Proceedings of the National Academy of Sciences of the United States of America: Vol. 106 Suppl 1 (Issue Supplement 1, pp. 9995-10000). National Academy of Sciences. https://doi.org/10.1073/pnas.0901522106.
108. Eid, J., Fehr, A., Gray, J., Luong, K., Lyle, J., Otto, G., Peluso, P., Rank, D., Baybayan, P., Bettman, B., Bibillo, A., Bjornson, K., Chaudhuri, B., Christians, F., Cicero, R., Clark, S., Dalal, R., DeWinter, A., Dixon, J., Turner, S. (2009). Real-time DNA sequencing from single polymerase molecules. Science, 323 (5910), 133-138. https://doi.org/10.1126/science. 1162986.

TABLE 2

| | | CONTROL | | | | INDUCED | |
|---|---|---|---|---|---|---|---|
| | | 0 | 12 | 24 | 72 | 0 | 12 |
| | | hours | hours | hours | hours | hours | hours |
| | Compound | M3-C1 | M3-C2 | M3-C3 | M3-C4 | M3-N1 | M3-N2 |
| | Hexadecanoic acid, trimethylsilyl ester | 23.894 | 27.276 | 2.6579 | 3.9565 | 21.473 | 20.0386 |
| | 1-Hexadecyne | n/d | n/d | | | n/d | 3.0843 |
| C16 TAG R | Hexadecanoic acid | 23.894 | 27.276 | 2.6579 | 3.9565 | 21.473 | 23.1229 |
| chain | | | | | | | |
| | Octadecanoic acid, trimethylsilyl ester | 4.5212 | 2.3482 | | | 5.3858 | 21.489 |
| | cis-13-Octadecenoic acid, trimethylsilyl ester | n/d | 1.2557 | | | 5.3858 | n/d |
| | Octadecanoic acid, trimethylsilyl ester | n/d | n/d | | | n/d | 2.3774 |
| | trans-13-Octadecenoic acid, trimethylsilyl | n/d | n/d | | | n/d | 1.2302 |
| | ester | | | | | | |
| C18 TAG R | Octadecanoic acid | 4.5212 | 3.6039 | n/d | n/d | 10.7716 | 25.0966 |
| chain | | | | | | | |
| C22 PUFA | cis-4,7,10,13,16,19-Docosahexaenoic acid | n/d | n/d | n/d | n/d | 1.2631 | n/d |
| C20 PUFA | 5,8,11,14,17-Eicosapentaenoic acid, | n/d | n/d | | | n/d | n/d |
| | methyl ester, (all-Z)- | | | | | | |
| | cis-5,8,11,14,17-Eicosapentaenoic acid, | n/d | 3.9508 | | | n/d | 4.2158 |
| | trimethylsilyl ester | | | | | | |
| | 1,4-Eicosadiene | 4.1458 | n/d | | | 6.6978 | n/d |
| | Eicosapentaenoic acid | 4.1458 | 3.9508 | n/d | n/d | 7.9609 | 4.2158 |
| | 9,12,15-Octadecatrienoic acid, | n/d | n/d | | | n/d | n/d |
| | methyl ester, (Z,Z,Z)- | | | | | | |
| | 9,12-Octadecadienoic acid, | n/d | n/d | | | n/d | n/d |
| | methyl ester | | | | | | |
| | 9,12-Octadecadienoic acid (Z,Z)-, | n/d | 16.1466 | | | 13.0735 | n/d |
| | trimethylsilyl ester | | | | | | |
| | 9,12-Octadecadienoic acid (Z,Z)-, | 10.8783 | n/d | | | n/d | n/d |
| | trimethylsilyl ester / Linoleic acid, TMS | | | | | | |
| | 9,12-Octadecadienoic acid (Z,Z)-, | 8.2839 | n/d | | | n/d | n/d |
| | trimethylsilyl ester / Linoleic acid, TMS | | | | | | |
| | .alpha.-Linolenic acid, trimethylsilyl ester | n/d | 8.5565 | | | 7.4041 | 9.4237 |
| C18 PUFA | Linolenic acid | 19.1622 | 24.7031 | n/d | n/d | 20.4776 | 9.4237 |
| Carotenoid | Bicyclo[3.1.1]heptane,2,6,6-trimethyl-, | 17.0007 | 1.1783 | n/d | n/d | 14.4784 | 7.389 |
| | (1.alpha.,2.beta.,5.alpha.)- | | | | | | |
| Sterol | B-Sitosterol | 4.8169 | 1.5635 | | | n/d | 3.608 |
| Chlorophyll | 3,7,11,15-Tetramethyl-2-hexadecen-1-ol | 5.9453 | n/d | | | n/d | n/d |
| | 3,7,11,15-Tetramethyl-2-hexadecen-1-ol | n/d | n/d | | | n/d | 1.8719 |
| | 3,7,11,15-Tetramethyl-2-hexadecen-1-ol | 5.9453 | n/d | n/d | n/d | n/d | 1.8719 |

| | | INDUCED | | | | |
|---|---|---|---|---|---|---|
| | | 24 | 72 | STRAIN COMPARISON | | |
| | | hours | hours | HB001 | HB002 | HB003 |
| | Compound | M3-N3 | M3-N4 | M1 | M2 | M3 |
| | Hexadecanoic acid, trimethylsilyl ester | 17.4641 | 18.5183 | 7.2186 | 23.593 | 16.359 |
| | 1-Hexadecyne | n/d | 3.7314 | n/d | | 3.0625 |
| C16 TAG R | Hexadecanoic acid | 17.4641 | 22.2497 | 7.2186 | 23.593 | 19.422 |
| chain | | | | | | |
| | Octadecanoic acid, trimethylsilyl ester | n/d | n/d | n/d | 3.650 | n/d |
| | cis-13-Octadecenoic acid, trimethylsilyl ester | n/d | n/d | n/d | | n/d |
| | Octadecanoic acid, trimethylsilyl ester | 1.9564 | 2.3167 | 4.7554 | | n/d |
| | trans-13-Octadecenoic acid, trimethylsilyl | 1.9076 | 1.4753 | 6.5132 | 2.652 | 3.1621 |
| | ester | | | | | |
| C18 TAG R | Octadecanoic acid | 3.864 | 3.792 | 11.269 | 6.302 | 3.1621 |
| chain | | | | | | |
| C22 PUFA | cis-4,7,10,13,16,19-Docosahexaenoic acid | 1.3664 | n/d | n/d | 2.142 | n/d |
| C20 PUFA | 5,8,11,14,17-Eicosapentaenoic acid, | n/d | 3.5906 | n/d | | 2.3065 |
| | methyl ester, (all-Z)- | | | | | |
| | cis-5,8,11,14,17-Eicosapentaenoic acid, | n/d | n/d | n/d | | n/d |
| | trimethylsilyl ester | | | | | |
| | 1,4-Eicosadiene | n/d | n/d | n/d | | n/d |
| | Eicosapentaenoic acid | 1.3664 | 3.5906 | n/d | n/d | 2.3065 |
| | 9,12,15-Octadecatrienoic acid, | n/d | n/d | n/d | | 4.2046 |
| | methyl ester, (Z,Z,Z)- | | | | | |
| | 9,12-Octadecadienoic acid, | n/d | n/d | n/d | | 4.4777 |
| | methyl ester | | | | | |
| | 9,12-Octadecadienoic acid (Z,Z)-, | 7.8546 | 18.6136 | 14.2001 | 13.8269 | 9.3542 |
| | trimethylsilyl ester | | | | | |
| | 9,12-Octadecadienoic acid (Z,Z)-, | n/d | n/d | n/d | | n/d |
| | trimethylsilyl ester / Linoleic acid, TMS | | | | | |
| | 9,12-Octadecadienoic acid (Z,Z)-, | n/d | n/d | n/d | | n/d |
| | trimethylsilyl ester / Linoleic acid, TMS | | | | | |
| | .alpha.-Linolenic acid, trimethylsilyl ester | 4.7322 | 9.579 | 10.4021 | | 7.5987 |
| C18 PUFA | Linolenic acid | 12.5868 | 28.1926 | 24.6022 | 13.8269 | 25.6352 |
| Carotenoid | Bicyclo[3.1.1]heptane,2,6,6-trimethyl-, | 1.8368 | 8.8428 | 7.1805 | 2.1954 | 8.6901 |
| | (1.alpha.,2.beta.,5.alpha.)- | | | | | |
| Sterol | B-Sitosterol | n/d | 3.2569 | 8.9878 | 1.0403 | 1.9294 |
| Chlorophyll | 3,7,11,15-Tetramethyl-2-hexadecen-1-ol | n/d | n/d | n/d | n/d | n/d |
| | 3,7,11,15-Tetramethyl-2-hexadecen-1-ol | n/d | n/d | 2.5808 | n/d | n/d |
| | 3,7,11,15-Tetramethyl-2-hexadecen-1-ol | n/d | n/d | 2.5808 | n/d | n/d |

TABLE 5

| Compound type | Low % | High % |
|---|---|---|
| Hexadecanoic acid (C16) | 17 | 23 |
| Octadecanoic acid (C18) | 3% | 25% |
| Eicosapentaenoic acid (C20) | 1% | 8% |
| Linolenic acid (C18) | 9% | 29% |
| Bicyclo[3.1.1]heptane, 2,6,6-trimethyl-, | 1% | 15% |
| (1.alpha.,2.beta.,5.alpha.)- | | |
| B-Sitosterol | <1% | 4% |
| 3,7,11,15-Tetramethyl-2-hexadecen-1-ol | <1% | 2% |

### Selected Sphaerica Triacylglycerides (>1% Peak Height) Structures

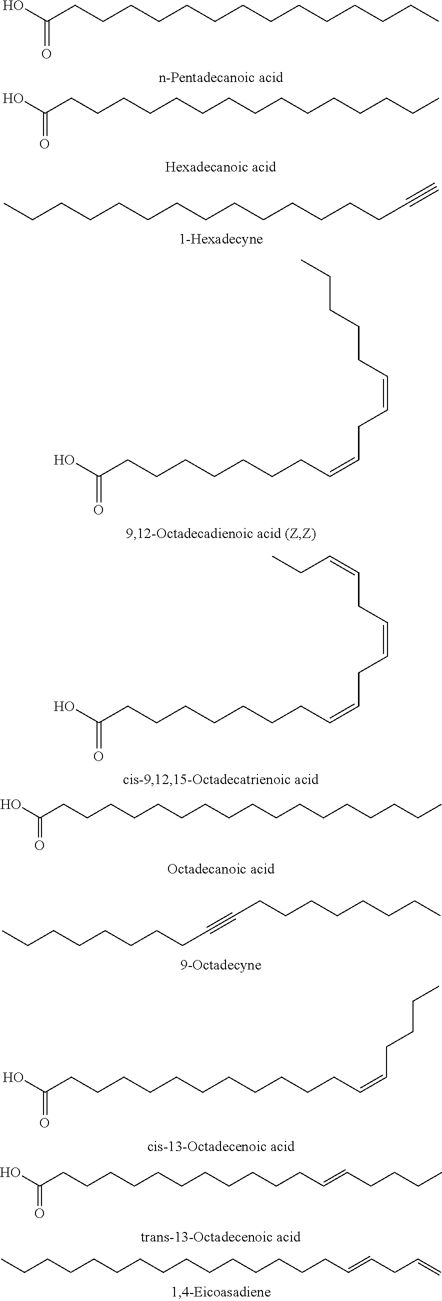

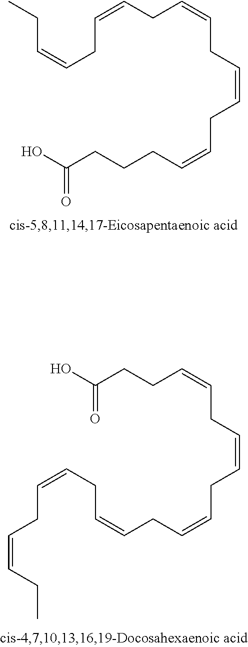 [SMILES: CC/C=C\C/C=C\C/C=C\C/C=C\C/C=C\CCCC(=O)O ; CC/C=C\C/C=C\C/C=C\C/C=C\C/C=C\C/C=C\CCC(=O)O]

1. A marine microalgal strain of the Ulvales order of the Ulvophyceae class characterised by at least 5, 6, 7, or 8 of the following properties:
(i) being unicellular and uni-nucleate;
(ii) non motile, without cilia nor flagellae;
(iii) having generally spherical cells;
(iv) having a diameter of about 1.00 to 11.0 μm, more preferably 1.5 μm to 10.3 μM;
(v) having an extracellular polysaccharide capsule 0.215 μm to 0.344 μm thick;
(vi) having chloroplasts that are parietal and a single pyrenoid;
(vii) includes lipid bodies.
(viii) capable of asexual reproduction within maternal capsule through two sequential divisions at approximately 90° to each other into 4 nonmotile daughter cells enclosed within their own capsule, daughter cells released by rupture of maternal cell envelope,
said strain also:
(ix) having from 120-300 thylakoid membranes per chloroplast
(x) having an 18S rRNA which exhibits 97% or higher base sequence homology with the 18S rRNA sequence shown in FIG. 5

2. A microalgal strain of claim 1 characterised by having all of features (i)-(x).

3. A microalgal strain of claim 1 or claim 2 wherein:
(i) the chloroplasts exhibit penta-radial symmetry occupying upper cell hemisphere with lobes extending into the remaining hemisphere which contains the lipid bodies; and/or
(ii) the thylakoids enclose an approximately 17 nm wide lumen and form a continuous structure throughout the chloroplast with thylakoids running parallel to the cell membrane;
and/or
(iii) the neutral lipid content is from 5% to 70%, optionally 5% to 65%, of cell dry mass.

4. A microalgal strain of any one of claims 1 to 3 which:
(i) has higher growth rates when cultured externally on a 12:12 day/night cycle having a maximum daytime insolation of greater than or equal to 1600 μM photons s⁻¹·m⁻² as compared to a maximum of 150, 200, 250, or 300 μM photons s⁻¹·m⁻²; and/or
(ii) remains viable after 72-hours culture at 45° C.

5. A microalgal strain as claimed in any one of claims 1 to 4 wherein the said strain is capable of producing the compounds shown in Table 2 and optionally Table 3.

6. A microalgal strain characterized in that it consists of an isolated strain deposited at the CCAP under accession number CCAP 2271/1, CCAP 2271/2 or CCAP 2271/3.

7. A microalgal strain as claimed in any one of claims 1 to 5 which strain is obtained or obtainable by screening from a marine environment using filtered seawater supplemented with vitamin-free nitrogen sources, phosphorous and trace elements.

8. A microalgal strain as claimed in claim 7 which strain is present in the same phylogenetic clade within the Ulvophyceae as a strain deposited under accession number CCAP 2271/1, CCAP 2271/2 or CCAP 2271/3.

9. A microalgal strain which strain is a derivative of any of a marine microalgal strain defined in any one of claims 6 to 8 having the properties of the microalgae defined in any one of claims 1 to 5.

10. A microalgal strain as claimed in claim 9 which is a directed evolution engineered derivative strain which is capable of more efficient growth in vitamin-free media having a PSU of greater than or equal to 30, 35, 40, or 45 PSU, as compared to the growth rate in otherwise identical low PSU media.

11. A strain as claimed in any one of claims 1 to 10 which is in the form of biologically pure culture of said strain, which may be a slope culture or liquid medium broth.

12. A strain as claimed in any one of claims 1 to 10 which is in the form of a freeze dried sample, a liquid nitrogen frozen sample, or a frozen preparation in glycerol of said strain

13. A cell extract; a cell suspension; a cell homogenate; a cell lysate; or a cell pellet of a strain as claimed in any one of claims 1 to 10.

14. A culture broth of, or a cell free or substantially cell free culture broth of, a strain as claimed in any one of claims 1 to 10.

15. A process for making a derived strain of claim 9 from the deposited strain of claim 6 or from the screened strain of claim 7, which process comprises directed evolution.

16. A process as claimed claim 15 wherein the directed evolution comprises one or more of the following steps:
(i) culturing the deposited parent strain under an environmental stress selected from: high salinity and/or high insolation, wherein the intensity of said stress is increased step-wise;
(ii) maintaining said culture over several generations;
(iii) isolating the strain(s) for which the number of cells has most increased during said generations.

17. A process for making a derived strain of claim 9 from the deposited strain of claim 6 or from the screened strain of claim 7, which process comprises mutagenesis and selection.

18. A process as claimed claim 17 wherein the mutagenesis and selection comprises one or more of the following steps:
(i) culturing the deposited parent strain under conditions promoting mutagenesis;
(ii) maintaining said culture over several generations;
(iii) isolating the strain(s) for which the number of cells has most increased during said generations.

19. A process for making a derived strain of claim 9 from the deposited strain of claim 6 or from the screened strain of claim 7 by recombinant molecular biology, comprising one or more of gene insertion, deletion, or modification.

20. A method of producing microalgal biomass or producing a desired product or compound produced by microalgal culture, which method comprises culturing a population of marine microalgae as claimed in any one of claims 1 to 12.

21. A method as claimed in claim 20 wherein said marine microalgae are cultured:
(i) at a temperature of between about 20° C. to about 50° C., preferably 28° C.-35° C.; and/or
(ii) at a pH of between 6 and 11, preferably about pH 8; and/or
(iii) a salinity of between 15 to 63 PSU, preferably 30 to 40 PSU; and/or
(iv) a photonic intensity of up to 2100 μmol photons m⁻²·s⁻¹ Photosynthetically Available Radiation.

22. A method as claimed in claim 20 or claim 21 wherein the marine microalgal cells are mixed during culture via air bubbling into the culture media and/or are supplemented with CO₂, which is optionally industrial flue gas.

23. A method as claimed in any one of claims 20 to 22 wherein the marine microalgae are cultured externally under natural day/night cycle, and wherein:
(i) the maximum light intensity in the peak day part of the cycle is at least or about 100 to 2100 μmol photons m⁻²·s⁻¹ at peak daytime exposure; and/or
(ii) the maximum temperature at the peak day part of the cycle is between 30° C. to about 50° C., optionally at least 35° C., 38° C., 48° C.; and/or
(ii) the maximum salinity during culture is at least or about 30, 35, 40, 45, 50, 55, 60 or 63 PSU.

24. A method as claimed in any one of claims 20 to 23 wherein the culture is photoautotrophic.

25. A method as claimed in any one of claims 20 to 24 wherein the culture is carried out in a culture medium which is a synthetic medium prepared from:
(i) either (ia) a natural source of salt-water, which optionally sea water, brackish water, or a salt-lake water, or (ib) an industrial brine product;
(ii) an additional source or sources of a nitrogen containing compound, which is optionally nitrate or urea, and phosphate;
(iii) optionally an additional source of trace elements; and
(iv) preferably no added vitamins nor added antibiotic nor antifungal supplements.

26. A method as claimed in claim 25 wherein an additional source of trace elements is provided, and optionally comprises one or more of: FeCl₃, CuSO₄, ZnSO₄, CoCl₂, MnCl₂, or Na₂MoO₄.

27. A method as claimed in claim 25 or claim 26 wherein the media comprises filtered sea water supplemented with NaNO₃ about 7 mM; NaH₂PO₄·2H₂O about 0.3 mM; C₁₀H₁₄N₂Na₂O₈·2H₂O about 20 μM; FeCl₃·6H₂O about 25 μM; CuSO₄·5H₂O about 29 nM; ZnSO₄·7H₂O about 150 nM; CoCl₂·6H₂O about 150 nM; MnCl₂·4H₂O about 2 μM; Na₂MoO₄·2H₂O about 50 nM.

28. A method as claimed in any one of claims 20 to 27 wherein the method is performed:
(i) as batch culture; or
(ii) continuous culture.

29. A method as claimed in any one of claims 20 to 28 wherein the method is performed:
(i) in an open pond, or
(ii) in a culture reactor, which is optionally a vertical culture reactor.

30. A method as claimed claim 29 wherein the method is performed in a culture reactor which is a photobioreactor, which is optionally an air lift column or flat panel.

31. A method as claimed in any one of claims 20 to 30 wherein the method is for producing a desired compound selected from:
(i) a biofuel comprising microalgal triacylglycerides; and/or
(ii) polyunsaturated fatty acids (PUFA); and/or
(iii) other lipid fractions; and/or (iv) high protein, PUFA rich food or fish- or animal-feed.

32. A method as claimed in any one of claims 20 to 31 which further comprises the step of recovering, isolating, purifying, or enriching the desired product from the culture.

33. A method as claimed in claim 32 wherein the desired product is derived from the supernatant of the culture, optionally by one or more of centrifugation, filtration or decanting, followed optionally by precipitation.

34. A method as claimed in claim 32 or claim 33 wherein the desired product is recovered by tangential flow filtration.

35. A method as claimed in claim 32 for producing a triacylglyceride composition from the culture, which method comprises solvent extraction of said triacylglycerides.

36. A method as claimed in claim 32 or claim 35 comprising the steps of:
(a) filtering the cell mass from the culture;
(b) lysing the algal cells in the cell mass in the presence of a polar-solvent, which is optionally methanol;
(c) mixing the polar-solvent and lysed cellular material with a non-polar solvent which is optionally hexane;
(d) recovering the triacylglyceride-containing non-polar solvent layer;
(e) recovered the triacylglyceride from the non-polar solvent layer,
or which comprises the steps of:
(a) filtering the cell mass from the culture;
(b) drying the cell mass;
(c) optionally disrupting/lysing cells in the cell mass;
(d) solubilising metabolic products such as triacylglycerides using super-critical CO2 which optionally may also utilise organic co-solvents.

37. A desired product obtained or obtainable by the method of any one of claims 32 to 36, which product is:
(i) a triacylglyceride enriched composition, or
(ii) a polyunsaturated fatty acids (PUFA) enriched composition

38. A PUFA composition as claimed in claim 37, optionally characterised in that it comprises at least Linolenic acid; Eicosapentaenoic acid; Arachidonic acid.

39. A triacylglyceride enriched composition as claimed in claim 37, optionally characterised in that:
(i) the most prevalent fatty acid types in the triacylglyceride are hexadecanoic acid and octadecadienoic acid; and/or
(ii) the composition comprises one or more, or all, of the following compounds: 1-Hexadecyne; cis-13-Octadecenoic acid; trans-13-Octadecenoic acid; 1,4-Eicosadiene; and/or
(iii) the composition does not comprise the following compounds: tetradecanoic acid; heptadecanoic acid; nonadecanoic acid.

40. A triacylglyceride enriched composition as claimed in claim 39 which further comprises: Bicyclo[3.1.1]heptane, 2,6,6-trimethyl-, (1.alpha.,2.beta.,5.alpha.); B-Sitosterol; 3,7,11,15-Tetramethyl-2-hexadecen-1-ol.

41. A triacylglyceride enriched composition as claimed in claim 39 or 40 characterised in that it comprises one or more, or all, of the compounds described in Table 5 within the respective % range shown therein.

42. A triacylglyceride enriched composition as claimed in any one of claims 39 to 41 characterised by a GC-MS profile substantially as shown in FIG. 14 or Table 3, or having a fatty acid composition substantially as shown in Table 2.

43. A process for producing a composition of fatty acid methyl esters, the process comprising transesterification of the triacylglyceride enriched composition of any one of claims 39 to 42.

44. A process for producing a fuel product, the process comprising catalytic hydrodeoxygenation of the triacylglyceride enriched composition of any one of claims 39 to 42.

45. A process for producing a refined product, the process comprising industrial refining of the triacylglyceride enriched composition of any one of claims 39 to 42, or the cultured microalgal biomass obtained by any one of claims 20 to 30.

46. A composition of fatty acid methyl esters obtained or obtainable by the process of claim 43.

47. A fuel product or refined product obtained or obtainable by the process of claim 44 or claim 45 respectively.

48. Use of the triacylglyceride enriched composition of any one of claims 39 to 42, methyl ester composition of claim 46 or fuel product or refined product of claim 47 as a fuel or as a fuel component.

49. A fuel, which is optionally an automobile, aviation or marine fuel, comprising a product as claimed in any one of claim 39 to 42, 46 or 47.

50. A biodiesel fuel comprising a product as claimed in any one of claims 39 to 42, optionally 5% to 24% of said product in combination with 95% to 76% standard diesel fuel or methyl ester composition of claim 46 or fuel product or refined product of claim 47.

51. A high protein agricultural feedstock comprising microalgal biomass obtained according to the method of any one of claims 20 to 30.

52. Use of as a microalgal strain as claimed in any one of claims 1 to 10, or a method of any one of claims 20 to 30, for carbon capture from a CO2 containing gas, which is optionally an industrial flue gas.
